(19) United States
(12) Patent Application Publication
(10) Pub. No.: US 2025/0011391 A1
(43) Pub. Date: Jan. 9, 2025

(54) ULTRA LIGHT-SENSITIVE NEUROPSIN-BASED OPTOGENETIC TOOL FOR ACTIVATING G q-COUPLED SIGNALING AND/OR ACTIVATING CELLS

(71) Applicant: GENANS BIOTECHNOLOGY CO., LTD, Changping District Beijing (CN)

(72) Inventors: Tao Yu, Changping District Beijing (CN); Ruicheng Dai, Changping District Beijing (CN); Danwei Weng, Changping District Beijing (CN); Minmin Luo, Changping District Beijing (CN)

(21) Appl. No.: 18/709,383

(22) PCT Filed: Dec. 20, 2022
(86) PCT No.: PCT/CN2022/140440

§ 371 Date: May 10, 2024

(30) Foreign Application Priority Data
Dec. 20, 2021 (WO) PCT/CN2021/139751

(51) Int. Cl. C07K 14/72; A61K 38/00; A61N 5/06; C12N 15/86

(52) U.S. Cl. CPC C07K 14/723; A61N 5/0618; A61N 5/062; C12N 15/86; A61K 38/00; A61N 2005/0662; C12N 2750/14143

(57) ABSTRACT

The present invention relates to an isolated light-sensitive opsin for rapidly, reversibly, and precisely activating G_{q} signaling and/or activating cells.

### REFERENCE TO SEQUENCE LISTING

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Apr. 11, 2024, is entitled “WO11697BSUS.xml”, and has a file size of 12,630 bytes.

### INTRODUCTION

G-protein-coupled receptors (GPCRs) modulate many intracellular signaling pathways and represent some of the most intensively studied drug targets (Hauser et al., 2017). Upon ligand binding, the GPCR undergoes a conformation change that is transmitted to heterotrimeric G proteins, which are multi-subunit complexes comprising G_{α} and tightly associated G_{βγ} subunits. The G_{q} proteins, a subfamily of heterotrimeric G_{α} subunits, couple to a class of GPCRs to mediate cellular responses to neurotransmitters, sensory stimuli, and hormones throughout the body. Their primary downstream signaling targets include phospholipase C beta (PLC-β) enzymes, which catalyze the hydrolysis of phospholipid phosphatidylinositol bisphosphate (PIP₂) into inositol trisphosphate (IP₃) and diacylglycerol (DAG). IP₃ triggers the release of Ca²⁺ from intracellular stores into the cytoplasm, and Ca²⁺ together with DAG activate protein kinase C (PKC). Several tools, including chemogenetics and photoactivatable small molecules, have been developed to study the signaling mechanisms and physiological functions of G_{q}-coupled GPCRs and intracellular Ca²⁺ release.

Optogenetics uses light-responsive proteins to achieve optically-controlled perturbation of cellular activities with genetic specificity and high spatiotemporal precision. Since the early discoveries of optogenetic tools using light-sensitive ion channels and transporters, diverse technologies have been developed and now support optical interventions into intracellular second messengers, protein interactions and degradation, and gene transcription. Opto-alAR, a creatively designed G_{q}-coupled rhodopsin-GPCR chimera, can induce intracellular Ca²⁺ increase in response to long-time photostimulation (60 s) (Airan et al., 2009). However, this tool has not been widely used, possibly because of its limitations associated with light sensitivity and response kinetics (Tichy et al., 2019). Most animals detect light using GPCR-based photoreceptors, which comprise both a protein moiety (opsin) and a vitamin A derivative (retinal) that functions as both a ligand and a chromophore. Several thousand opsins have been identified to date. Two recent studies, having reported Gᵢ-based opsins from mosquito and lamprey for presynaptic terminals inhibition in neurons, elegantly demonstrated that some naturally occurring photoreceptors are suitable for use as efficient optogenetic tools. Regarding the G_{q} signaling, melanopsin (Opn4) in a subset of mammalian retinal ganglion cells is a G_{q}-coupled opsin that mediates no-image-forming visual functions. However, HEK293 or Neuro-2a cells heterologously expressing Opn4 showed weak light responses and required additional retinal in the culture medium. Opn5 (neuropsin) and its orthologs in many vertebrates have been reported as an ultraviolet (UV)-sensitive opsin that couples to Gᵢ proteins.

### SUMMARY OF THE INVENTION

The present invention relates to an isolated light-sensitive opsin for rapidly, reversibly, and precisely activating G_{q} signaling and/or activating cells.

In the first place, the present invention relates to an isolated light-sensitive opsin for activating G_{q} signaling and/or activating cells.

In some embodiments, the light has a wavelength ranging range of 360 nm-520 nm, preferably, 450-500, more preferably, 460-480 nm.

In some embodiments, the isolated opsin is an isolated opsin from an organism, its homologs, its orthologs, its paralogs, fragments or variants thereof having the activity of activating G_{q} signaling and/or activating cells.

In some embodiments, the isolated opsin shares at least 70%, at least 80%, at least 90%, at least 95%, at least 96%, at least 97%, at least 98%, or at least 99% identity to the wild type opsin in the organism, its homologs, its orthologs, its paralogs, fragments or variants thereof, and has the activity of activating G_{q} signaling and/or activating cells.

In some embodiments, the organism is an animal.

In some embodiments, the isolated opsin is an isolated opsin 5(Opn5) from an animal, its homologs, its orthologs, its paralogs, fragments or variants thereof having the activity of activating G_{q} signaling and/or activating cells.

In some embodiments, the isolated opsin 5(Opn5) shares at least 70%, at least 80%, at least 90%, at least 95%, at least 96%, at least 97%, at least 98%, or at least 99% identity to the wild type opsin 5(Opn5) in the animal, its homologs, its orthologs, its paralogs, fragments or variants thereof, and has the activity of activating G_{q} signaling and/or activating cells.

In some embodiments, the animal is a vertebrate animal.

In some embodiments, the animal is an avian, a reptile, or a fish, an amphibian, or a mammal.

In some embodiments, the animal is an avian, including but not limited to chicken, duck, goose, ostrich, emu, rhea, kiwi, cassowary, turkey, quail, chicken, falcon, eagle, hawk, pigeon, parakeet, cockatoo, makaw, parrot, perching bird (such as, song bird), jay, blackbird, finch, warbler and sparrow.

In some embodiments, the animal is a reptile including but not limited to lizard, snake, alligator, turtle, crocodile, and tortoise.

In some embodiments, the animal is a fish including but not limited to catfish, eels, sharks, and swordfish.

In some embodiments, the animal is an amphibian including but not limited to a toad, frog, newt, and salamander.

In some embodiments, the isolated opsin 5(Opn5) is an isolated wild type opsin 5(Opn5) from the chicken, or fragments or variants thereof having the activity of activating G_{q} signaling and/or activating cells.

In some embodiments, the isolated opsin 5(Opn5) shares at least 70%, at least 80%, at least 90%, at least 95%, at least 96%, at least 97%, at least 98%, or at least 99% identity to the wild type opsin 5(Opn5) from the chicken, and has the activity of activating G_{q} signaling and/or activating cells.

In some embodiments, the isolated opsin 5(Opn5) is an isolated wild type opsin 5(Opn5) from the turtle, or fragments or variants thereof having the activity of activating G_{q} signaling and/or activating cells.

In some embodiments, the isolated opsin 5(Opn5) shares at least 70%, at least 80%, at least 90%, at least 95%, at least 96%, at least 97%, at least 98%, or at least 99% identity to the wild type opsin 5(Opn5) from the turtle, and has the activity of activating G_{q} signaling and/or activating cells.

In some embodiments, the isolated opsin 5(Opn5) has the amino acid sequence shown by SEQ ID NO:1 (cOpn5), or fragments or variants thereof having the activity of activating G_{q} signaling and/or activating cells.

In some embodiments, the isolated opsin 5(Opn5) shares at least 70%, at least 80%, at least 90%, at least 95%, at least 96%, at least 97%, at least 98%, or at least 99% identity to the amino acid sequence shown by SEQ ID NO:1 (cOpn5), and has the activity of activating G_{q} signaling and/or activating cells.

In some embodiments, the isolated opsin 5(Opn5) has the amino acid sequence shown by SEQ ID NO:2 (tOpn5), or fragments or variants thereof having the activity of activating G_{q} signaling and/or activating cells.

In some embodiments, the isolated opsin 5(Opn5) shares at least 70%, at least 80%, at least 90%, at least 95%, at least 96%, at least 97%, at least 98%, or at least 99% identity to the amino acid sequence shown by SEQ ID NO:2 (tOpn5), and has the activity of activating G_{q} signaling and/or activating cells.

The isolated opsin 5(Opn5) may be used as a convenient optogenetic tool that precisely activates intracellular G_{q} signaling and/or activating cells.

In the second place, the present invention relates to an isolated nucleic acid encoding the isolated opsin in the first place.

In some embodiments, the isolated nucleic acid encodes the wild type opsin in the organism, its homologs, its orthologs, its paralogs, fragments or variants thereof having the activity of activating G_{q} signaling and/or activating cells.

In the third place, the present invention relates to a chimeric gene comprising the sequence of the isolated nucleic acid in the second place operably linked to suitable regulatory sequences.

In the fourth place, the present invention relates to a vector comprising the isolated nucleic acid in the second place, or the chimeric gene in the third place.

The vector is a eukaryotic vector, a prokaryotic expression vector, a viral vector, or a yeast vector.

In some embodiments, the vector is a herpes virus simplex vector, a vaccinia virus vector, or an adenoviral vector, an adeno-associated viral vector, a retroviral vector, or an insect vector.

Preferably, the vector is a recombinant AAV1, AAV2, AAV3, AAV4, AAV5, AAV6, AAV7, AAV8, AAV9, AAVS, AAVO or AAV10.

In some embodiments, the vector is an expression vector.

In some embodiments, the vector is a gene therapy vector.

In the fifth place, the present invention relates to an isolated cell or a cell culture, comprising the isolated nucleic acid in the second place, the chimeric gene in the third place, or the vector in the fourth place.

For example, expressing cOpn5 in HEK 293T cells powerfully mediates blue light-triggered, G_{q}-dependent Ca²⁺ increase from intracellular stores.

For example, optogenetic activation of cOpn5-expressing astrocytes induces massive ATP release in the mouse brain.

In the sixth place, the present invention relates to use of the isolated opsin in the first place, the isolated nucleic acid in the second place, the chimeric gene in the third place, the vector in the fourth place, or the isolated cell or the cell culture in the fifth place for treating a disease or a condition mediated by, or involving activating G_{q} signaling and/or activating cells.

cOpn5-mediated optogenetics can be applied to activate neurons and control animal behavior in a circuit-dependent manner.

In the seventh place, the present invention relates to a method of treating a disease or condition mediated by or involving activating G_{q} signaling and/or activating cells in a subject, comprising administering the isolated opsin in the first place, the isolated nucleic acid in the second place, the chimeric gene in the third place, the vector in the fourth place, or the isolated cell or the cell culture in the fifth place.

In some embodiments, the disease or condition mediated by or involving activating G_{q} signaling and/or activating cells includes but not limited to diseases or conditions benefiting from activating G_{q} signaling and/or activating cells, for example, benefiting from the activation of astrocytes, strong ATP release, or elevating neuron activity.

In some embodiments, the disease or condition mediated by or involving activating G_{q} signaling and/or activating cells includes but not limited to diseases or conditions benefiting from activating cells, such as islet cells, immune cells, nerve cells, for example, central neurons, astrocytes, glial cells, muscle cells, skeletal cells, endothelial cells, epithelial cells, nervous system cells, skin cells, lung cells, kidney cells and liver cells, cardiac cells, or vascular endothelial cells.

In some embodiments, the disease or condition includes but not limited to diabetes, immunosuppressive disease, Alzheimer's disease, depression, anxiety neurosis, cerebral haemorrhage, and so on.

In some embodiments, the method further comprises applying blue light having a wavelength range of 360 nm-520 nm, preferably, 450-500, more preferably, 460-480 nm.

In some embodiments, the method further comprises applying two-photon activation using long-wavelength (≥920 nm) light.

The isolated opsin in the present invention is sensitive to the light having a wavelength ranging 360-550 nm, preferably, 450-500, more preferably, 460-480 nm. In particular, 470 nm blue light elicits the strongest Ca² transients in cells, which means that the isolated opsin in the present invention is ultra-sensitive to the light a wavelength of 470 nm.

The invention encompasses all combination of the particular embodiments recited herein.

### BRIEF DESCRIPTION OF THE DRAWINGS

FIG. 1 shows that cOpn5 mediates light-induced strong activation of G_{q} signaling in HEK 293T cells.

FIG. 2 shows that cOpn5 couples to G_{q} but not Gᵢ signaling.

FIG. 3 shows that cOpn5 sensitively mediates optical control of G_{q} signaling with high temporal and spatial resolution.

FIG. 4 shows that cOpn5 mediates more rapid and sensitive response to light than opto-a1AR, hM3Dq or opn4.

FIG. 5 shows that cOpn5 effectively mediates the activation of astrocytes.

FIG. 6 shows that cOpn5-mediated activation of astrocytes induces massive ATP flashes and neuron activation in vivo.

FIG. 7 shows that cOpn5 mediates persistent, reliable ATP release in astrocytes and activation of surrounding neurons.

FIG. 8 shows that cOpn5-mediated optogenetics changes mouse behaviors in a neural circuit-dependent manner.

FIG. 9 shows that cOpn5-mediated optogenetics reliably activates neurons.

FIG. 10 shows injection sites and the placement of optical fibers.

### DESCRIPTION OF PARTICULAR EMBODIMENTS OF THE INVENTION

In the present invention, the capacity of opsin, in particular, Opn5 orthologs from multiple species is tested and it is found that many opsins sensitively and strongly mediated light-induced activation of G_{q} signaling and/or activating cells.

Preferably, the Opn5 orthologs is chicken ortholog (cOpn5 for simplicity), or turtle ortholog (tOpn5 for simplicity).

Detailed characterizations of Opn5, in particular, cOpn5 reveal that it is super sensitivity to blue light (μW/mm²-level, ˜3 orders of magnitude more sensitive than existing G_{q}-coupled opsin-based tools: opto-a1AR and opn4), high temporal (in response to 10 ms light pulses, ˜3 orders of magnitude more rapidly than opto-alAR or opn4) and spatial (subcellular level) resolution, and no need of chromophore addition. In particular, endogenous retinal is sufficient and no retinal is needed to be added.

The present invention further demonstrates cOpn5 optogenetics as a highly effective approach for activating astrocytes to induce massive ATP release in vivo, as well as for activating neurons to produce robust behavior changes in freely moving mice. These findings establish that cOpn5, and potentially other Opn5 orthologs, can be utilized as powerful optogenetic tools to support experimental investigations into the physiological and behavioral functions associated with G_{q} signaling and/or activating cells in both non-excitable cells and excitable cells.

cOpn5 Mediates Optogenetic Activation of G_{q} Signaling and/or Activating Cells.

Specifically, in the present invention, Opn5 orthologs from chicken, turtles, humans and mice (which share 80-90% protein sequence identity from each other) are tested in order to determine whether they have the capacity to mediate blue light-induced G_{q} signaling activation within HEK 293T cells. Blue light for stimulation and the red intracellular calcium indicator Calbryte™ 630 AM dye are used to monitor the relative Ca²⁺ response. It is found that the Opn5 orthologs from chicken (cOpn5) and turtle (tOpn5) mediated an immediate and strong light-induced increase in Ca²⁺ signal (˜3 ΔF/F), whereas no light effect is observed from cells expressing the human or mouse Opn5 orthologs. As exemplified by the chicken ortholog, the cOpn5 co-localized with the EGFP-CAAX membrane marker, indicating that it is efficiently transported to the plasma membrane. No exogenous retinal is needed to be added to the culture media, which suggests that endogenous retinal is sufficient to render cOpn5 functional. The Ca²⁺ signals are resistant to the removal of extracellular Ca²⁺, thus indicating Ca²⁺ release from the intracellular stores. Preincubation of G_{q} proteins inhibitor, for example, YM-254890, a highly selective G_{q} proteins inhibitor, reversibly abolished the light-induced Ca²⁺ transients in both cOpn5-expressing cells. In cOpn5-, but not human OPN5-expressing cells, a light-induced increase in the level of inositol phosphate (IP1), the rapid degradation product of IP3, is detected; moreover, the extent of this increase is reduced with the treatment of YM-254890. In cOpn5-expressing cells, for example, HEK 293T cells, blue light also triggers the phosphorylation of MARCKS protein, a well-established target of PKC, in a PKC activity-dependent manner. By contrast, blue light illumination effectively reduces cAMP levels in cells expressing human and mouse Opn5 with retinal, but has no such effect in cells expressing cOpn5 without retinal. Collectively, these data support that blue light illumination enables the coupling of cOpn5 to the G_{q} signaling pathway in HEK 293T cells.

cOpn5-Mediated Optogenetics is Sensitive and Precise.

Specifically, the light-activating properties of cOpn5 are characterized in the present invention. cOpn5 may be heterologously expressed in cells, for example, in HEK 293T cells. Although Opn5 is previously considered as an ultraviolet (UV)-sensitive photoreceptor, mapping with a set of wavelengths ranging 365-630 nm at a fixed light intensity of (100 μW/mm2) reveals that the 470 nm blue light elicits the strongest Ca²⁺ transients, with the UVA light (365 and 395 nm) being less effective and longer-wavelength visible light (561 nm or above) completely ineffective. The effects of different light durations on cOpn5-expressing HEK 293T cells are tested, and stimulating with brief light pulses (1, 5, 10, 20, 50 ms; 16 μW/mm2; 470 nm) shows that the Ca²⁺ response achieves the saturation mode with light duration over 10 ms. Longer light durations do not further increase the Ca²⁺ signal amplitude at this light intensity (16 μW/mm²; 470 nm). Delivering 470 nm light at different intensities shows that blue light of ˜4.8 μW/mm² and 16 μW/mm² produce about half maximum and full maximum responses, respectively. These data suggest that the light sensitivity of cOpn5 is 2-3 orders of magnitude higher than the reported values of the commonly used optogenetic tool Channelrhodopsin-2 (ChR2). Together, the results in the present invention indicate that cOpn5 could function as a single-component optogenetic tool without additional retinal, and that cOpn5 is super-sensitive to blue light for its full activation requiring low light intensity (16 μW/mm²) and short duration (10 ms).

The performance of cOpn5 to that of opto-a1AR, a chimera GPCR engineered by mixing rhodopsin with G_{q}-coupled adrenergic receptor is compared. Following the protocol in a previous report, it is found that very long exposure of strong illumination (60 s; 7 mW/mm²) is required to trigger a slow and small (˜0.5 ΔF/F) Ca²⁺ signal increase in opto-a1AR-expressing HEK 293T cells, and 15 s illumination is inefficient. The performance of cOpn5 to that of opn4, a natural opsin which is reported as a tool for G_{q} signaling activating is compared. It is found that long exposure of strong illumination (25 s; 40 mW/mm²) and additional retinal is required to trigger a slow (˜1 ΔF/F) Ca²⁺ signal increase in opn4-expressing HEK 293T cells. Therefore, compared with existing opsin-based tools (opto-a1AR and opn4), cOpn5 is much more light-sensitive (˜3 orders more sensitive), requires much shorter time exposure (10 ms vs. 60 s), and produces stronger responses.

Furthermore, the performance of cOpn5 to that of the popular G_{q}-coupled chemogenetic tool hM3Dq, which is activated by adding the exogenous small molecule ligand clozapine-N-oxide (CNO) is compared. Light-induced activation of cOpn5-expressing HEK 293T cells has a similar peak response amplitude of the Ca²⁺ signal as CNO-induced activation of hM3Dq-expressing HEK 293T cells. Meanwhile, cOpn5-expressing HEK 293T cells has faster and temporally more precise response, as well as more rapid recovery time than hM3Dq-expressing HEK 293T cells. These results indicate that cOpn5-mediated optogenetics are more controllable in temporal accuracy than those of hM3Dq.

cOpn5 optogenetics allows spatially precise control of cellular activity. Restricting brief light stimulation (63 ms) into a subcellular region of individual cOpn5-expressing HEK 293T cell results in the immediate activation of a single cell. Interestingly, in high cell confluence area, Ca²⁺ signals propagate to surrounding cells, thus suggesting intercellular communication among HEK 293T cells through a yet-to-identified mechanism. cOpn5 is expressed in primary astrocyte cultures prepared from the neonatal mouse brain with AAV vectors for bicistronic expression of cOpn5 and the EGFP marker protein. Using the Calbryte 630 AM dye to monitor Ca²⁺ levels, it is found that blue light illumination of cOpn5-expressing astrocytes produces strong Ca²⁺ transients (˜8 ΔF/F). When the light stimulation (63 ms) is precisely restricted to only subcellular region of an individual cOpn5-expressing astrocyte, it is observed Ca²⁺ signal propagation within the individual cell. Resembling the tests in HEK 293T cells, wave-like propagation of Ca²⁺ signals from the stimulated astrocyte that proceeded gradually to more distal, non-stimulated, astrocytes, is observed. These experiments thus demonstrate that cOpn5 optogenetics allows precise spatial control, and suggest that it may be useful to study the dynamics of astrocytic networks, which was initially discovered using neurochemical and mechanical stimulation.

cOpn5 Optogenetic Activation of Astrocytes Induces Massive ATP Release and Neuron Activation In Vivo.

The performance of cOpn5-mediated optogenetics in vivo is tested. Astrocytes represent an important population of non-excitable cells in the central nervous system, over which optogenetic tools have achieved only limited success to date. ATP is known as a messenger for inter-astrocyte communication; however, the real-time impacts of intracellular Ca²⁺ on ATP release have not been visualized. The recently-reported ultrasensitive GPCR Activation-Based ATP sensor GRAB_{ATP} is employed to monitor changes in extracellular ATP levels. Specifically, cOpn5 and the GRAB_{ATP} sensor are expressed in the mouse S1 sensory cortex following the infusion of AAV vectors containing the GfaABC1D promoter, which is commonly used to drive gene expression in astrocytes.

Two-photon imaging of GRAB_{ATP} signals from head-fixed awake, behaving mice, is performed. Strikingly, the 920 nm light itself, delivers for imaging, triggers massive ATP flashes in the cOpn5- and GRABATP-expressing mice, but not in mice that express the ATP sensor but lack cOpn5 expression. Blue light pulses are not required to stimulate ATP signals. Individual ATP flashes typically range in diameters of 20-100 μm and last for ˜1 min. The flash frequency gradually increases following ˜1 min of initial quiescence and peaks at the level of ˜50 flashes per min within the imaging area (640×640 μm²) in ˜5 min. Moreover, high-frequency ATP flashes also occurred in the repeated trials. In mice expressing GRAB_{ATP} alone, sporadic ATP events (˜0.3 flashes per min within the imaging area) are observed; eight hours following the proinflammatory treatment of intraperitoneal lipopolysaccharide (LPS) injections, the ATP flash events increased nearly 6 fold of the basal condition (˜2 flashes per min) but exhibited rather stable frequency, which confirmed that inflammation induces ATP release in the brain. Given that the observed ATP flash frequency in cOpn5-expressing mice was ˜25 times more than that by the proinflammatory treatment of non-cOpn5-expressing mice, it is demonstrated that cOpn5-mediated light activation of astrocytes induces continuous massive ATP release and in vivo.

Astrocytes release ATP and other gliotransmitters also act on neuronal receptors to modulate neuron activity. Two-photon imaging of neuronal Ca²⁺ signals with cOpn5-mediated astrocytes activation from head-fixed awake, behaving mice, is performed. cOpn5-expressing cells (n=406) colocalize with GFAP-staining (n=397), but not GCaMP7b-expressing neurons. Raw trace examples and group data show that, compared the 15-20 min to 0-5 min, cOpn5-mediated astrocytes activation significantly elevated neuron activity. cOpn5 strictly expresses in astrocytes that demonstrated by the colocalization of cOpn5-expressing cells and GFAP staining signals which differ from GCaMP7b signals of neurons. It is demonstrated that cOpn5-mediated light activation of astrocytes elevates the activities of surrounding neurons in vivo. Moreover, the present invention shows that the long-wavelength (920 nm) light from pulsed laser for two-photon imaging is able to activate cOpn5, indicating the possibility of two-photon optogenetics for cOpn5.

cOpn5 Optogenetics Activates Neurons and Modulates Animal Behaviors.

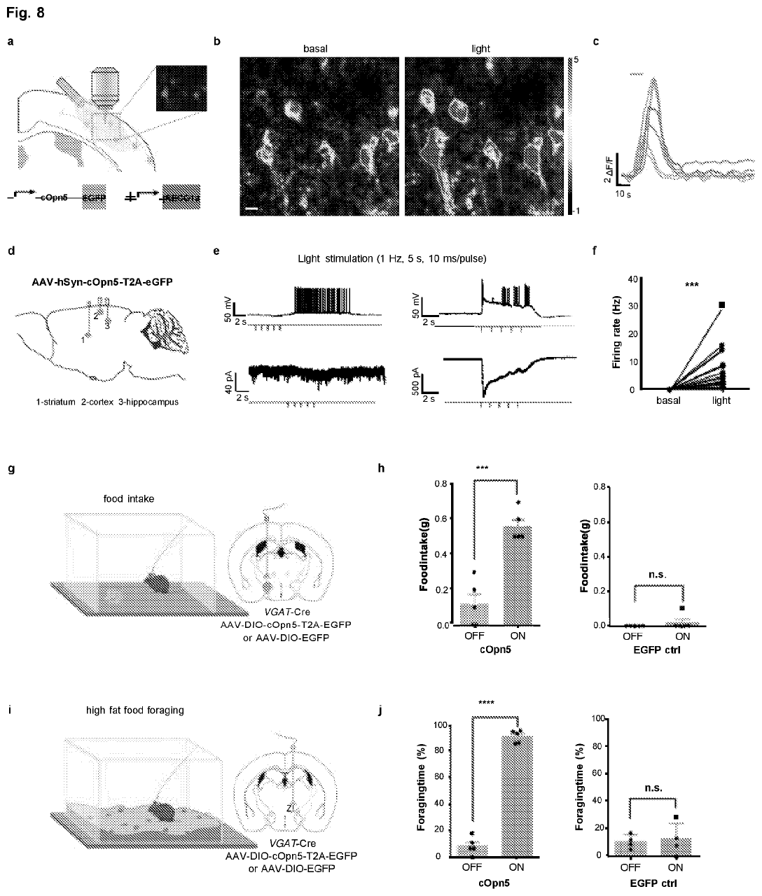

The application of cOpn5-mediated optogenetics in neurons is explored. Whether cOpn5 could mediate light-induced Ca²⁺ signals is firstly examined. Using AAV and the pan-neuronal SYN promoter, cOpn5 and the genetically-encoded Ca²⁺ sensor jRGECOla is expressed in mouse cortical neurons (FIG. 8a). In brain slice preparations, application of blue light pulses (10 s; 100 μW/mm²; 473 nm) reliably evoked Ca²⁺ transients in neurons (FIG. 8b, c). Thus, cOpn5 also enables light-induced activation in neurons.

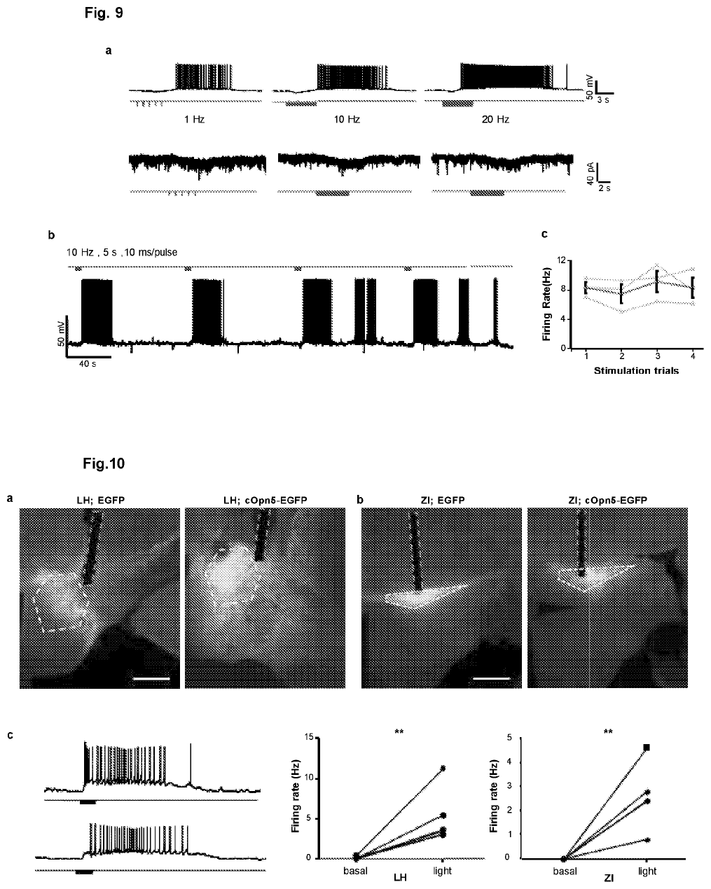

The effect of light-induced cOpn5 activation on the electrophysiological properties of neurons in slice preparations of the motor cortex, hippocampus, and dorsal striatum is investigated (FIG. 8d). Two types of activation patterns are observed. In a majority of recorded neurons, blue light pulses induce a small depolarizing current (˜20 pA) in the voltage-clamp mode, and induce delayed-yet-vigorous firing of action potentials in the current-clamp mode (FIG. 8e, left, n=12 neurons). At higher frequencies of light pulses, cOpn5 drove more spikes with shorter latency (from ˜5 s to ˜3 s) after the initial light pulse, while the inward current is not significantly affected (FIG. 9a). In the other subset of neurons, brief light pulses rapidly evoked strong inward currents (100-1000 pA) and drove bursting firing of action potentials (FIG. 8e, right, n=6 neurons). Neurons were repeatedly stimulated with 10 ms/pulse at 10 Hz, and exhibited a non-attenuated mode in firing rate across repetitive trials of light stimulation (FIG. 9b). Of note, unlike generated by ChR2 optogenetics, the action potential evoked by cOpn5 photostimulation was not time-locked to light pulses.

Finally, the utility of cOpn5-mediated optogenetics for modulating animal behavior is assessed. The lateral hypothalamus (LH) is a brain center with known functions in reward processing and feeding behaviors. We expressed cOpn5 in the LH GABAergic neurons of VGAT-Cre mice and implanted optical fibers to deliver light pulses into the LH of freely behaving mice (FIG. 8g). Consistent with previous findings that activating LH GABA neurons drives feeding behavior 56, light stimulation (20 Hz; 5 ms/pulse; 473 nm; 0.75 mW output from the fiber tip) elicited a significant increase in food intake in cOpn5-expressing mice but not the EGFP-expressing control mice (FIG. 8h). A food-foraging behavior task is also used to test the effect of cOpn5-mediated optogenetic activation of GABA neurons in the zona incerta (ZI) (FIG. 8i), a region known to drive compulsive eating. cOpn5-expressing mice, comparing with the EGFP-expressing mice, show a significantly increase in the time of foraging high fat food pellets upon repeated light stimulation (FIG. 8j). Electrophysiological recordings on LH and ZI cOpn5-expressing neurons are performed to characterize the response profiles. The injection sites and placement of optical fibers are confirmed by whole brain slices (FIG. 10a-c). Notably, mice maintained the behavior (feeding behavior or high-fat food foraging behavior) while the light was on, and immediately stopped the behavior when the light was off. Thus, cOpn5 is effective for rapidly, accurately, and reversibly modulating animal behavioral states.

Here, the present invention demonstrates the use of Opn5 of the present invention as an extremely effective optogenetic tool for activating G_{q} signaling and/or activating cells. Previous studies have characterized mammalian Opn5 as a UV-sensitive Gᵢ-coupled opsin; we present the surprising finding that visible blue light can induce rapid Ca²⁺ transients, IP₁ accumulation, and PKC activation in Opn5-expressing, for example cOpn5-expressing or tOpn5-expressing mammalian cells. The present invention Opn5 in the present invention, in particular, cOpn5 in mouse astrocytes effectively mediates light-evoked strong ATP release and elevates neuron activity in vivo. The present invention also shows that Opn5, in particular, cOpn5 allows rapid, robust, and reversible optical activation of neurons and applies it for the selective modulation of animal behavior. Importantly, Opn5 in the present invention, for example, cOpn5 is a powerful yet easy-to-use, single-component system that does not require an exogenous chromophore. The present invention envisions that Opn5-based optogenetics, for example, cOpn5-based optogenetics will be an enabling technique for investigating the important physiological and behavioral functions regulated by G_{q}-coupled signaling and/or activating cells in both non-excitable and excitable cells.

Table 6 lists the enabling features of cOpn5 by directly comparing its response amplitudes, light sensitivity, temporal resolution, and the requirement of additional chromophores to those of other optogenetic tools. For cOpn5-expressing cells, merely 10 ms blue light pulses at the intensity of 16 μW/mm² evoke rapid increase in Ca²⁺ signals with the peak amplitudes of 3-8 ΔF/F. By contrast, prior to the present invention, it is revealed that the activation of opto-a1AR or mammalian Opn4, the two proposed optogenetic tools for G_{q} signaling, require ˜3-fold higher light intensity (7-40 mW/mm²) and prolonged light exposure (20-60 s) and produce only weak Ca²⁺ signals (0.25-0.5 ΔF/F). Therefore, opto-a1AR or mammalian Opn4 cannot mimic the rapid activation profiles of endogenous G_{q}-coupled receptors that often trigger strong G_{q} signaling upon subsecond application of their corresponding ligands. We demonstrate the power of cOpn5 optogenetics by showing the striking physiological and behavioral effects in response to cOpn5-mediated optical activation of astrocytes and neurons in vivo. By contrast, recent systematic characterizations show that opto-a1AR- and Opn4-mediated optogenetic stimulations do not increase the amplitudes of Ca²⁺ signals and only mildly modulate the frequency of Ca²⁺ transients and synaptic events even after prolonged illumination (Gerasimov et al., 2021; Mederos et al., 2019). By overcoming the limitations of light sensitivity, temporal resolution, and response amplitudes associated with opto-a1AR- and Opn4-mediated optogenetics, Opn5 in the present invention, in particular, cOpn5 or tOpn5 will find broad applicability in studying the mechanisms and functions of G_{q} signaling and/or activating cells in numerous cells and tissues.

Opn5 in the present invention, in particular, cOpn5 or tOpn5-based optogenetics also enjoys the benefit of safety and convenience. Although Opn5 from many species are reported UV-responsive (Kojima et al., 2011), cOpn5 is optimally activated by 470 nm blue light, which penetrates better than UV and avoids UV-associated cellular toxicity. Its ultra-sensitivity to light also minimizes potential heating artifact. It is two-photon activable using long-wavelength (≥920 nm) light, suggesting it is suitable for even deeper tissue activation using a pulsed laser. cOpn5 or tOpn5 is strongly, and repetitively activated by light without the requirement for exogenous retinal, possibly because cOpn5 or tOpn5 is a bistable opsin that covalently binds to endogenous retinal and is thus resistant to photo bleaching (Koyanagi and Terakita, 2014; Tsukamoto and Terakita, 2010). By contrast, mammalian experiments of Opn4 requires additional retinal and have long response time and low light sensitivity. Opn5 in the present invention, in particular, cOpn5 or tOpn5 as a single-component system is particularly useful for in vivo studies as it avoids the burden of delivering a compound into the tissue during the experiment.

Opn5 optogenetics in the present invention, in particular, cOpn5 or tOpn5 optogenetics also offers some major advantages over chemogenetics and uncaging tools. It is temporally much more precise and offers single-cell or even subcellular spatial resolution. Although CNO/hM3Dq-mediated chemogenetics has been used to investigate the physiological and behavioral functions of non-excitable cells, such as astrocytes in the brain (Agulhon et al., 2013; Shen et al., 2021), its use in vivo typically requires many minutes for CNO to reach its target cells and tissues. The diffusive nature of compounds indicates that it is nearly impossible to chemogenetically stimulate G_{q} signaling with cellular and subcellular resolution. Opn5 in the present invention, in particular, cOpn5 or tOpn5 also differs from caged compound-based ‘uncaging’ tools such as caged calcium and caged IP3, since these tools require compound preloading and only partially mimic the Ca²⁺-related pathways associated with G_{q} signaling and/or activating cells. There exists other ‘uncaging’ tools, such as caged glutamate and caged ATP (Ellis-Davies, 2007; Lezmy et al., 2021), that target endogenous GPCRs. However, these caged compounds require their introduction into extracellular medium or the intracellular cytoplasm, which limits their applications in behaving animals (Adams and Tsien, 1993b). The advantages of Opn5 in the present invention, in particular, cOpn5 or tOpn5 over ‘uncaging’ tools are clearly demonstrated by experiments that Opn5 in the present invention, in particular, cOpn5 or tOpn5 is similar to be used as conveniently as the existing ChR2-based tools for modulating animal behaviors.

Opn5 in the present invention, in particular, cOpn5 or tOpn5, optogenetics should be particularly useful for precisely activating intracellular G_{q} signaling and/or activating cells, which subsequently triggers Ca²⁺ release from intracellular stores and activates PKC. Opn5 in the present invention, in particular, cOpn5 or tOpn5, differs from current channel-based optogenetic tools, such as ChR2 or its variants, which translocate cations across the plasma membrane. By controlling cellular membrane potentials and thus action potential firing, ChR2 and its variants have contributed tremendously to dissecting neural circuits; however, their successes have been more constrained in studying non-excitable cells that lack active ion channels for generating action potentials (Gourine et al., 2010). In addition to the applications on non-excitable cells, Opn5 in the present invention, in particular, cOpn5 or tOpn5, optogenetics can also stimulate G_{q} signaling in neurons and/or activating neurons and control animal behavior in a circuit-dependent manner. Of note, G_{q}-coupled GPCRs may promiscuously recruit G proteins and affect variable downstream signaling in a receptor- and cell-specific manner. Indeed, we observed that same light illumination parameters produce different activation patterns among different neurons. Opn5 in the present invention, in particular, cOpn5 or tOpn5-mediated optogenetic activation does not generate strictly time-locked action potential firing as precisely as that by ChR2 in neurons. This may be useful, since it avoids artificially synchronized neuronal activation. However, if temporally precise control of action potential firing is necessary, we recommend ion channel-based optogenetic tools.

In addition to the technical advances, our findings also have several functional implications on the signaling and/or activating of astrocytes in vivo. Although ATP is considered an important gliotransmitter, previous studies have revealed multiple releasing mechanisms that depend on the methods of treatments, the presence of extracellular Ca²⁺, and the exact form of cell and tissue preparations (Figueiredo et al., 2014; Hamilton and Attwell, 2010). Moreover, the presence of ATP release is often monitored indirectly. It has remained unclear whether activating astrocytes triggers ATP release, and if so, how this release is manifested in vivo. Here we provide the first demonstration that stimulating G_{q} signaling pathway within astrocytes triggers massive ATP release in the form of ATP flashes. Opn5 in the present invention, in particular, cOpn5 or tOpn5 optogenetics thus provides an ideal technique to study the molecular and cellular mechanisms underlying ATP release. In addition to ATP, astrocyte activation leads to the release of other gliotransmitters, such as D-serine, glutamate, and GABA. ATP can also be converted to other metabolites, such as adenosine. The gliotransmitters and their metabolites can exert complex modulatory effects on neuronal excitability and synaptic strength. For example, while ATP can activate neurons through various P2X and P2Y receptors, adenosine strongly inhibits neurons through A1 receptors (Lezmy et al., 2021; Zhang et al., 2003). It has remained unclear how these various effects are integrated to modulate neuronal activity in vivo. Here we reveal that optogenetic activation of cOpn5-expressing astrocytes significantly excite neurons in the S1 cortex of mice. Opn5 in the present invention, in particular, cOpn5 or tOpn5 optogenetics thus lays the foundation for dissecting the molecular, cellular, and circuit mechanisms underlying the complex interactions between astrocytes and neurons. In vivo experiments have also demonstrated that Opn5 in the present invention, in particular, cOpn5 or tOpn5, has compatibility with optogenetic probes and imaging sensors, such as genetically encoded Ca² indicators and GPCR-based neurotransmitter sensors. Opn5 in the present invention, in particular, cOpn5 or tOpn5, together with these sensors potentially allow an all-optical approach to transiently activate G_{q} signaling and/or activating cells and simultaneously monitor the relevant effects.

In summary, the present invention demonstrates Opn5 in the present invention, in particular, cOpn5 or tOpn5, as a blue light-sensitive opsin for rapidly, reversibly, and precisely activating G_{q} signaling and/or activating cells. The present invention also establishes Opn5 in the present invention, in particular, cOpn5 or tOpn5, as a powerful and easy-to-use optogenetic tool for activating both non-excitable cells and neurons. Given the importance of G_{q}-coupled GPCRs, it is expect that cOpn5 will find broad applications for dissecting the mechanisms and functions of G_{q} signaling in all major cell types and tissues.

The descriptions of particular embodiments and examples are provided by way of illustration and not by way of limitation. Those skilled in the art will readily recognize a variety of noncritical parameters that could be changed or modified to yield essentially similar results.

### EXAMPLES

### Materials and Methods

TABLE 1
Primers for cloning

| | |
|---|---|
| V5-cOpn5 forward primer | 5′-cgtgaggtaccggatcctctagaatgggcaagcccatccccaacc |
| | ccctgctgggcctggacagcaccatgagtgggatggcatcggac-3′ |
| | (SEQ ID NO: 3) |
| V5-cOpn5 reverse primer | 5′-tcgataagcttgatatcgaattcttagacttccagttgggttccgct-3′ |
| | (SEQ ID NO: 4) |
| cOpn5-T2A-eGFP for hSyn | 5′-tagagtcgagctcaagcttgccaccatgagtgggatggcatcggactgca-3′ |
| promoter forward primer | (SEQ ID NO: 5) |
| cOpn5-T2A-eGFP for hSyn | 5′-aaccgcgggccctctagagcatatgttacttgtacagctcgtccatgccg-3′ |
| promoter reverse primer | (SEQ ID NO: 6) |
| cOpn5-T2A-eGFP for GfaABC1D | 5′-acctccgctgctcgcggggtctagaatgagtgggatggcatcggactgca-3′ |
| promoter forward primer | (SEQ ID NO: 7) |
| cOpn5-T2A-eGFP for GfaABC1D | 5′-tatcgataagcttgatatcgaattcttacttgtacagctcgtccatgccg-3′ |
| promoter reverse primer | (SEQ ID NO: 8) |
| cOpn5-T2A-eGFP for EF1a | 5′-tacattatacgaagttatggcgcgccttattacttgtacagctcgtccatg-3′ |
| promoter forward primer | (SEQ ID NO: 9) |
| cOpn5-T2A-eGFP for EF1a | 5′-atactttatacgaagttatgctagccaccatgagtgggatggcatcggactg-3′ |
| promoter reverse primer | (SEQ ID NO: 10) |
| cOpn5-T2A-mCherry forward | 5′-gcatcacctccgctgctcgcggggtatgagtgggatggcatcggactgca-3′ |
| primer | (SEQ ID NO: 11) |
| cOpn5-T2A-mCherry reverse | 5′-tcaccatggtggcgaccgggggatctgggccaggattctcctcgacgtca-3′ |
| primer | (SEQ ID NO: 12) |

TABLE 2
Recombinant DNA

| | |
|---|---|
| pcDNA3.1-opto-a1AR-EYFP | Addgene |
| | plasmid #20947 |
| EGFP-CAAX | Gift from Yulong Li |
| pLJM1-EGFP | Addgene |
| | plasmid #19319 |
| pAAV-GfaABC1D-hM3D(Gq)-mCherry | Addgene |
| | Plasmid #50478 |
| pAAV-EF1a-DIO-eGFP-WPRE-pA | N/A |
| pAAV-hSyn-GOI | N/A |
| pLJM1-cmv-cOpn5 | N/A |
| pLJM1-cmv-tOpn5 | N/A |
| pLJM1-cmv-hOPN5 | N/A |
| pLJM1-cmv-mOpn5 | N/A |
| pLJM1-cmv-V5-Opn5 | N/A |
| pLJM1-cmv-cOpn5-T2A-eGFP | N/A |
| PAAV-hSyn-cOpn5-T2A-eGFP-WPR-pA | N/A |
| PAAV-GfaABC1D-cOpn5-T2A-eGFP-WPR-pA | N/A |
| pAAV-EF1a-DIO-cOpn5-T2A-eGFP-WPRE-pA | N/A |
| PAAV-GfaABC1D-cOpn5-T2A-mCherry-WPR-pA | N/A |

TABLE 3
Virus Strains

| | |
|---|---|
| Lenti-cmv-cOpn5-puro | Chinese Institute for Brain Research, Beijing |
| Lenti-cmv-hOPN5-puro | Chinese Institute for Brain Research, Beijing |
| Lenti-cmv-tOpn5-puro | Chinese Institute for Brain Research, Beijing |
| Lenti-cmv-mOpn5-puro | Chinese Institute for Brain Research, Beijing |
| Lenti-cmv-hM3Dq -puro | Chinese Institute for Brain Research, Beijing |
| AAV2/9-EF1a-DIO-cOpn5-T2A-eGFP | Chinese Institute for Brain Research, Beijing |
| AAV2/9-hSyn-cOpn5-T2A-eGFP | Chinese Institute for Brain Research, Beijing |
| AAV2/9-Ef1a-DIO-cOpn5-T2A-eGFP | Chinese Institute for Brain Research, Beijing |
| AAV2/8-GFaABC1D-cOpn5-T2A-eGFP | Chinese Institute for Brain Research, Beijing |
| AAV2/8-GfaABC1D-cOpn5-T2A-mCherry | Chinese Institute for Brain Research, Beijing |
| AAV2/9-EF1a-EGFP | Chinese Institute for Brain Research, Beijing |
| AAV2-EF1α-DIO-GCaMP6m | Chinese Institute for Brain Research, Beijing |
| AAV2/9-GfaABC1D-ATP1.0 | WZ Biosciences Inc. Cat. # YL006003-AV9 |
| AAV9-hSyn-NES-jRGECO1a-WPRE | WZ Biosciences Inc. Cat. # BS8-NOAAAV9 |
| AAV2/9-mCaMKIIa-jGCaMP7b-WPRE-pA | Shanghai Taitool Bioscience Co., Ltd Cat. # S0712-9-H20 |

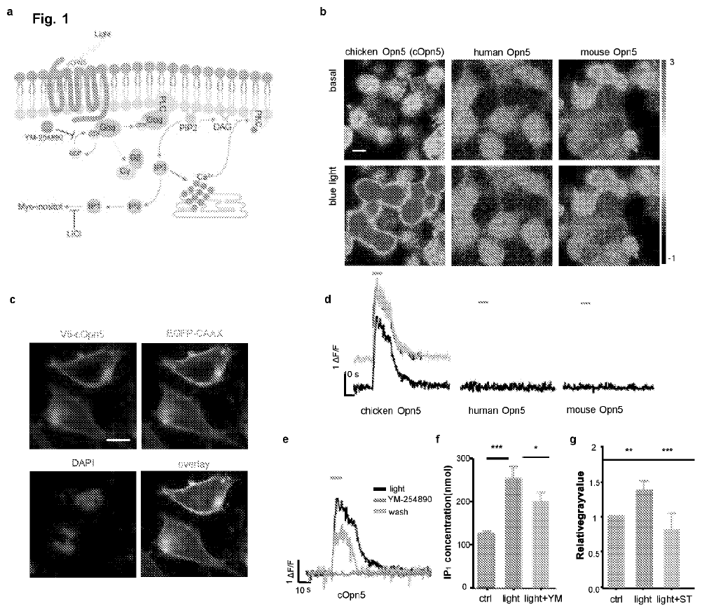

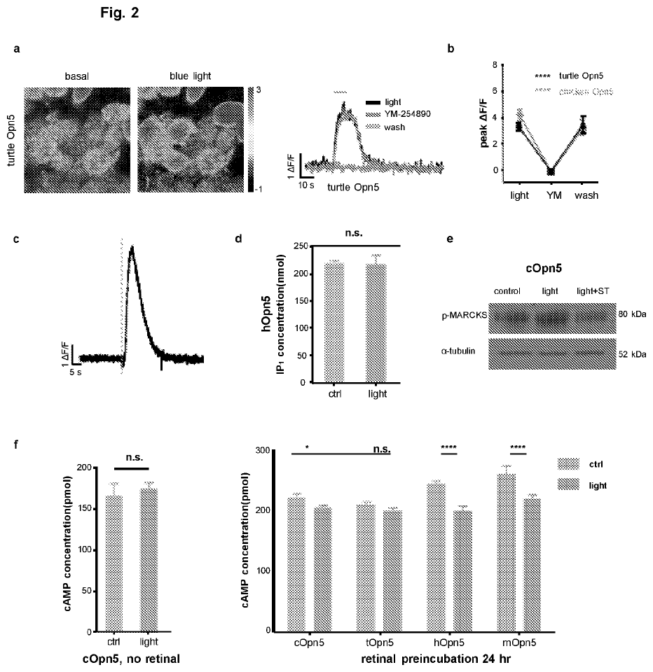

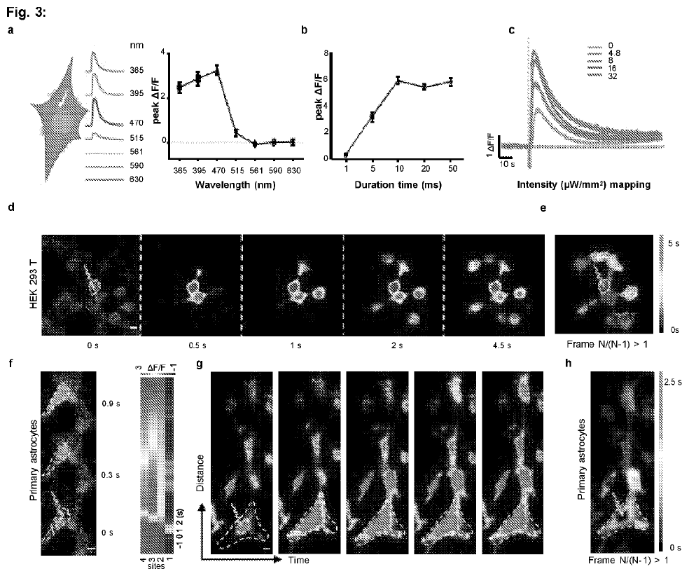

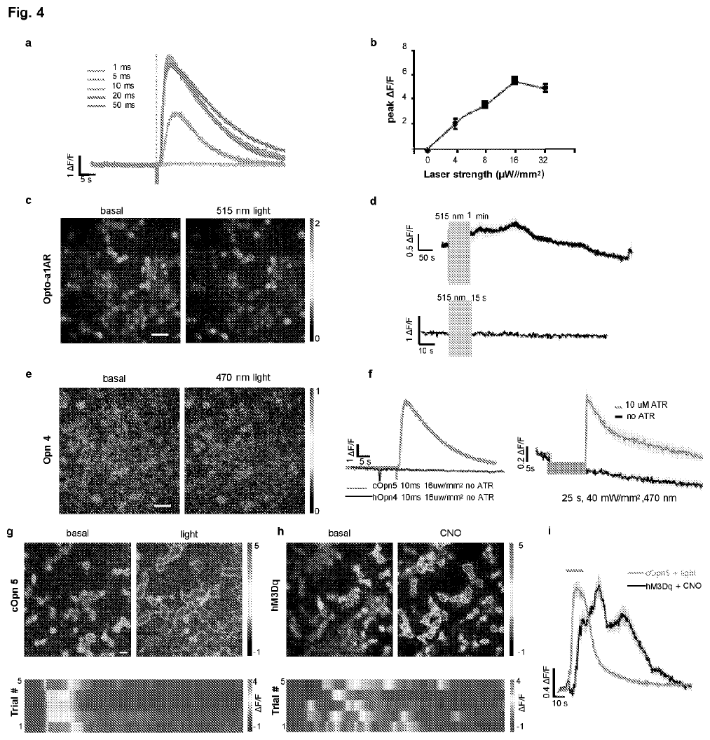

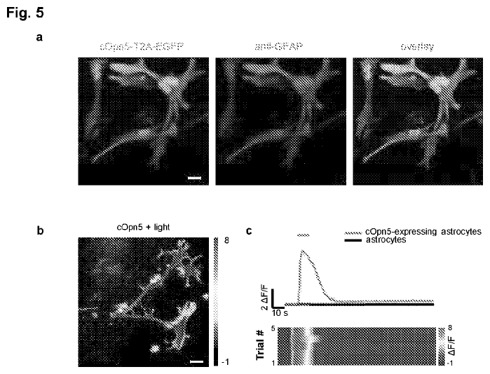

TABLE 4
Light excitation sources

| | | | |
|---|---|---|---|
| FIGS. 1f, 1g, FIGS. 2c, 2d, | 470 nm mounted LED | Thorlabs | M470L3 |
| 2e, 2f FIGS. 3b, 3c; FIGS. | | | |
| 4a, 4b, 4e, 4f, 4h | | | |
| FIGS. 5a, 5e, 5f, 5g, 8b | | | |
| FIGS. 1b, 1d, 1e; FIGS. | 488 nm from microscope | Nikon | A1R MP |
| 2a, 2b FIGS. 3d, 3f, 3g; | light source | | |
| FIG. 3a | 365 nm mounted LED | LG3535 | wavelength coverage: |
| | | | 360-370 nm |
| FIG. 3a | 395 nm mounted LED | LG3535 | wavelength coverage: |
| | | | 390-400 nm |
| FIG. 3a | 561 nm laser | Changchun New Industries | MGL-FN-561 |
| | | Optoelectronics Technology, China | |
| FIG. 3a | 590 nm mounted LED | CREE XP-E2 | wavelength coverage: |
| | | | 570-615 nm |
| FIG. 3a | 630 nm mounted LED | CREE XP-E2 | wavelength coverage: |
| | | | 615-660 nm |
| FIGS. 4c, 4d | 515 nm laser | Changchun New Industries | MGL-F-515 |
| | | Optoelectronics Technology, China | |

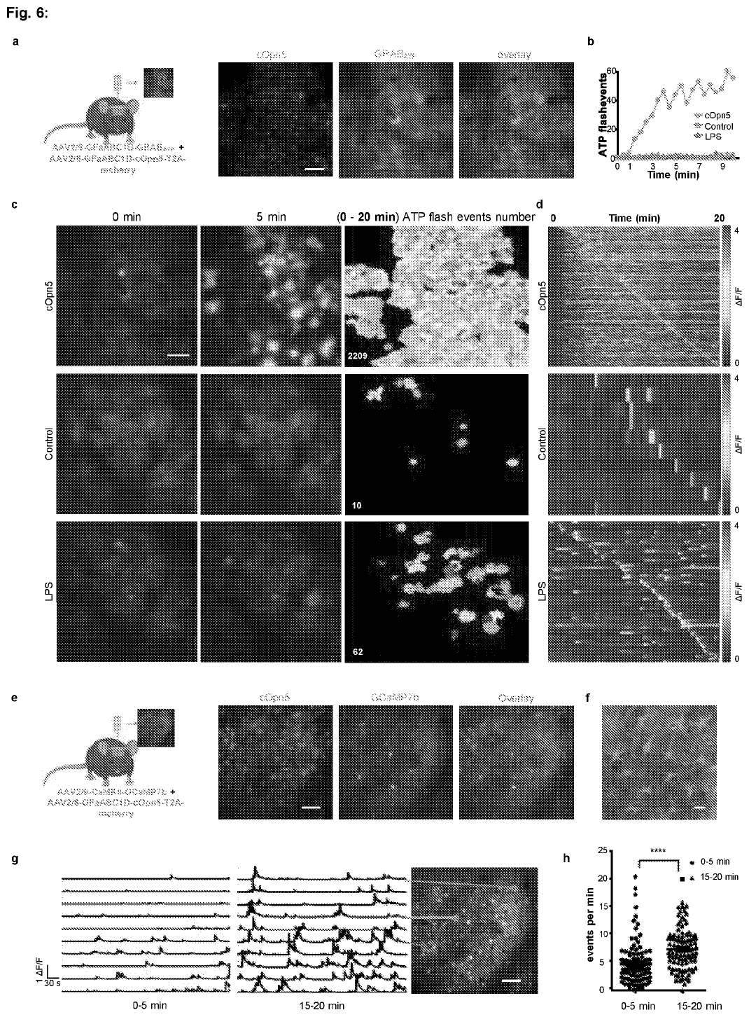

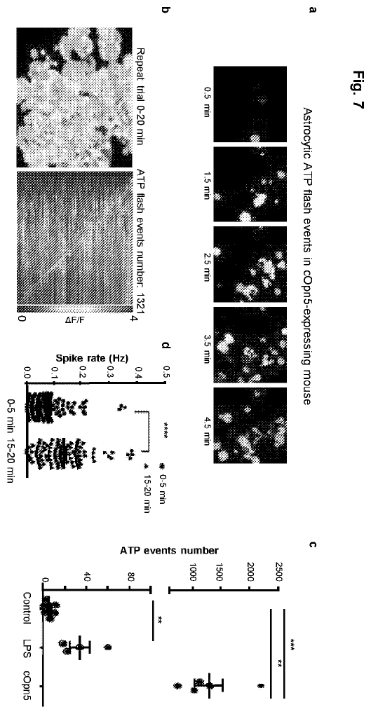

TABLE 5
Microscope equipments

| | | | |
|---|---|---|---|
| FIGS. 1b, 1d, 1e; FIGS. 2a, 2b | Multiphoton confocal microscopes | Nikon | A1R MP |
| FIGS. 3a, 3b, 3c; | Spinning Disk | Nikon | ECLIPASE Ti |
| FIG. 4a, 4b, 4c, 4d, 4e, 4f | | | |
| FIGS. 8a, 8b, 8c | Two-photon microscope | Olympus | BX61W1 |
| FIG. 5a | Confocal laser scanning microscope | Zeiss | LSM 880 |
| FIG. 6, FIG. 7 | Multiphoton microscope | Olympus | FluoView FVMPE- |
| | | | RS |

TABLE 6
Statistical analysis

| | | n per | | |
|---|---|---|---|---|
| FIG. | Conditions | group | Analysis | P value |
| 1f | ctrl vs. light | 4, 4 | Tukey's multiple comparisons test | P < 0.0001 |
| | light vs. | 4, 4 | Tukey's multiple comparisons test | P = 0.0128 |
| | light + YM-254890 | | | |
| 1g | ctrl vs. light | 4, 4 | Tukey's multiple comparisons test | P = 0.0096 |
| | light vs. | 4, 4 | Tukey's multiple comparisons test | P = 0.0004 |
| | light + staurosporine | | | |
| 6h | events per min: | 193, 193 | Unpaired t test | P < 0.0001 |
| | 0-5 min vs. 15-20 min | | | |
| 7d | Spike rate: | 193, 193 | Unpaired t test | P < 0.0001 |
| | 0-5 min vs. 15-20 min | | | |
| 8f | Basal vs. light | 18, 18 | Unpaired t test | P = 0.0005 |
| 8h | LH^{cOpn5} group: | 5, 5 | Unpaired t test | P = 0.0003 |
| | light on vs. off | | | |
| | LH^{EGFP} group: | 5, 5 | Unpaired t test | P = 0.3466 |
| | light on vs. off | | | |
| 8j | ZI^{cOpn5} group: | 5, 5 | Unpaired t test | P < 0.0001 |
| | light on vs. off | | | |
| | ZI^{EGFP} group: | 4, 4 | Unpaired t test | P = 0.7608 |
| | light on vs. off | | | |
| 2b | cOpn5 group: | 19, 15 | Tukey's multiple comparisons test | P < 0.0001 |
| | light vs YM-254890 | | | |
| | cOpn5 group: | 15, 11 | Tukey's multiple comparisons test | P < 0.0001 |
| | YM-254890 vs wash | | | |
| | cOpn5 group: | 19, 11 | Tukey's multiple comparisons test | P = 0.2239 |
| | light vs wash | | | |
| | tOpn5 group: | 15, 17 | Tukey's multiple comparisons test | P < 0.0001 |
| | light vs YM-254890 | | | |
| | tOpn5 group: | 17, 13 | Tukey's multiple comparisons test | P < 0.0001 |
| | YM-254890 vs wash | | | |
| | tOpn5 group: | 15, 13 | Tukey's multiple comparisons test | P = 0.9388 |
| | light vs wash | | | |
| 2d | ctrl vs. light | 4, 4 | Unpaired t test | P = 0.4338 |
| 2f- left | ctrl vs. light | 3, 3 | Tukey's multiple comparisons test | P = 0.992 |
| 2f- Right | cOpn5 group: | 4, 4 | Sidak's multiple comparisons test | P = 0.0223 |
| | ctrl vs. light | | | |
| | tOpn5 group: | 4, 4 | Sidak's multiple comparisons test | P = 0.4174 |
| | ctrl vs. light | | | |
| | hOPN5 group: | 4, 4 | Sidak's multiple comparisons test | P < 0.0001 |
| | ctrl vs. light | | | |
| | mOpn5 group: | 4, 4 | Sidak's multiple comparisons test | P < 0.0001 |
| | ctrl vs. light | | | |
| 7c | Basal vs. LPS | 6, 4 | Unpaired t test | P = 0.0066 |
| | Basal vs. Opn5 | 6, 2 | Unpaired t test | P = 0.0002 |
| | LPS vs. Opn5 | 4, 2 | Unpaired t test | P = 0.0031 |

### Example 1 cOpn5 Mediates Optogenetic Activation of G_{q} Signaling

Whether heterologous expression of the Opn5 orthologs from chicken, turtles, humans and mice (which share 80-90% protein sequence identity) have the capacity to mediate blue light-induced G_{q} signaling activation within HEK 293T cells is tested (FIG. 1a and table 7). Blue light for stimulation and the red intracellular calcium indicator Calbryte™ 630 AM dye are used to monitor the relative Ca²⁺ response (FIG. 1b). The Opn5 orthologs from chicken (cOpn5) and turtle (tOpn5) mediated an immediate and strong light-induced increase in Ca²⁺ signal (˜3 ΔF/F), whereas no light effect was observed from cells expressing the human or mouse Opn5 orthologs (FIG. 1d and FIG. 2a, b). As exemplified by the chicken ortholog, the cOpn5 co-localized with the EGFP-CAAX membrane marker, indicating that it was efficiently transported to the plasma membrane (FIG. 1c). No exogenous retinal is supplied to the culture media, which suggests that endogenous retinal is sufficient to render cOpn5 functional. The Ca²⁺ signals are resistant to the removal of extracellular Ca²⁺, thus indicating Ca²⁺ release from the intracellular stores (FIG. 2c). Preincubation of YM-254890, a highly selective G_{q} proteins inhibitor 33, reversibly abolishes the light-induced Ca²⁺ transients in both cOpn5-expressing cells (FIG. 1e). In cOpn5-, but not human OPN5-expressing cells, a light-induced increase in the level of inositol phosphate (IP₁), the rapid degradation product of IP₃ is detected; moreover, the extent of this increase is reduced with the treatment of YM-254890 (FIG. 1f and FIG. 2d). In cOpn5-expressing HEK 293T cells, blue light also triggers the phosphorylation of MARCKS protein, a well-established target of PKC 34, in a PKC activity-dependent manner (FIG. 1g and FIG. 2e). By contrast, blue light illumination effectively reduces cAMP levels in cells expressing human and mouse Opn5 with retinal, but has no such effect in cells expressing cOpn5 without retinal (FIG. 2f). Collectively, these data support that blue light illumination enables the coupling of cOpn5 to the G_{q} signaling pathway in HEK 293T cells.

TABLE 7
Opsins and species

| | Alias | species | |
|---|---|---|---|
| Chicken Opn5 | cOpn5 | Gallus gallus | GenBank |
| | | | NM_001130743.1 |
| Turtle Opn5 | tOpn5 | Chelonia mydas | GenBank |
| | | | XM_007068312.4 |
| Human Opn5 | hOPN5 | Homo sapiens | GenBank |
| | | | AY377391.1 |
| Mouse Opn5 | mOpn5 | Mus musculus | GenBank |
| | | | NM_181753.4 |

FIG. 1 Shows that cOpn5 Mediates Light-Induced Strong Activation of G_{q} Signaling in HEK 293T cells.

a, Schematic diagram of the putative intracellular signaling in response to light-induced cOpn5 activation. PLC: phospholipase C; PIP2: phosphatidylinositol-4,5-bisphosphate; IP₃: inositol-1,4,5-trisphosphate; IP₁: inositol monophosphate; DAG: diacylglycerol; PKC: protein kinase C; YM-254890: a selective G_{q} protein inhibitor.

b, Pseudocolor images of the Ca2+ signal before and after blue light stimulation (10 s; 100 μW/mm²; 488 nm) in HEK 293T cells expressing Opn5 from three species (Gallus gallus, Homo sapiens, and Mus musculus). Scale bar, 10 μm.

c, The Cy3-counterstained V5-cOpn5 fusion protein (red) was co-localized with the membrane-tagged EGFP-CAAX (green) in HEK 293T cells. DAPI counterstaining (blue) indicates cell nuclei. Scale bar, 10 μm.

d, Time courses of light-evoked Ca²⁺ signals for cells shown in c.

e, G_{q} protein inhibitor YM-254890 (10 nM) reversibly blocked cOpn5-mediated, light-induced Ca²⁺ signals.

f, YM suppressed the IP₁ accumulation evoked by continuous light stimulation (3 min; 100 μW/mm²; 470 nm) in cOpn5-expressing HEK 293T cells (Left). ***P<0.0001, *P=0.0128; Tukey's multiple comparisons test.

g, Phosphorylation of MARCKS in cOpn5-expressing HEK 293T cells in the control group (no light stimulation), the light stimulation group, and light+staurosporine (ST, PKC inhibitor) group. The amount of p-MARCKS in the same fraction was normalized to the amount of α-tubulin. **P=0.0096, ***P=0.0004; Tukey's multiple comparisons test.

FIG. 2 Shows that cOpn5 Couples to G_{q} but not Gᵢ Signaling

a, Pseudocolor images of the Ca2+ signal before and after blue light stimulation (10 s; 100 μW/mm2; 488 nm) in HEK 293T cells expressing Opn5 from turtle species (Chelonia mydas). Scale bar, 10 μm (left); Time courses of light-evoked Ca2+ signals for responded cells (right).

b, Group data of the G_{q} protein inhibitor YM-254890 (10 nM) reversibly blocked cOpn5- and turtle Opn5-mediated, light-induced Ca2+ signals. ****P<0.0001, one way ANOVA. Error bars indicate S.E.M.

c, Time course of Ca2+ signal with photostimulation (10 ms; 16 μW/mm2; 470 nm) without extracellular Ca2+.

d, IP1 accumulation in human Opn5-expressing HEK 293T cells with or without light stimulation (Right). n.s., no significant difference; unpaired t test.

e, One representative of phosphorylation of MARCKS in cOpn5-expressing HEK 293T cells in the control group (no light stimulation), the light stimulation group, and light+staurosporine group. The amount of p-MARCKS in the same fraction was normalized to the amount of α-tubulin.

f, Light has no effect on cAMP levels (10 μM forskolin preincubation) in cOpn5-expressing HEK 293T cells without additional retinal in the medium (left panel). Right panel shows the effects of photostimulation on cAMP concentrations for HEK 293T cells expressing Opn5s from four different species following 10 μM retinal preincubation.

Error bars in d and f indicate S.E.M.

### Example 2 cOpn5-Mediated Optogenetics is Sensitive and Precise

Characterizing the light-activating properties of cOpn5 heterologously expressed in HEK 293T cells is performed. Although Opn5 is previously considered as an ultraviolet (UV)-sensitive photoreceptor²⁷, mapping with a set of wavelengths ranging 365-630 nm at a fixed light intensity of (100 μW/mm²) revealed that the 470 nm blue light elicited the strongest Ca²⁺ transients, with the UVA light (365 and 395 nm) being less effective and longer-wavelength visible light (561 nm or above) completely ineffective (FIG. 3a). The effects of different light durations on cOpn5-expressing HEK 293T cells are tested. Stimulating with brief light pulses (1, 5, 10, 20, 50 ms; 16 μW/mm²; 470 nm) shows that the Ca²⁺ response achieves the saturation mode with light duration over 10 ms (FIG. 3b). Longer light durations do not further increase the Ca²⁺ signal amplitude at this light intensity (16 μW/mm²; 470 nm) (FIG. 4a). Delivering 470 nm light at different intensities shows that blue light of ˜4.8 μW/mm² and 16 μW/mm² produce about half maximum and full maximum responses, respectively (FIG. 3c and FIG. 4b). Therefore, the light sensitivity of cOpn5 is 3-4 orders of magnitude higher than the reported values of the light-sensitive G_{q}-coupled GPCRs and even 2-3 orders higher than those of the commonly used optogenetic tool Channelrhodopsin-2 (ChR2)(Lin, 2011; Zhang et al., 2006) (table 8). Together, these results indicate that cOpn5 could function as a single-component optogenetic tool without additional retinal, and tat cOpn5 is super-sensitive to blue light for its full activation requiring low light intensity (16 μW/mm²) and short duration (10 ms).

TABLE 8
Comparison cOpn5 with other optogenetic tools

| | | | | | | Need for | | |
|---|---|---|---|---|---|---|---|---|
| | Wavelength | Light | | Stimulation | | exogeneous | Response | |
| | λₘₐₓ (nm) | Sensitivity | | duration | | chemicals(retinal) | amplitude | model |
| Wild-type | 470 nm | 8-12 | mW/mm² | 2.3 ± 1.1 | ms | No | steady state:peak | Hippocampal |
| ChR2 ^{1,2} | | | | | | | current ratio:0.4 ± | cell culture |
| | | | | | | | 0.04; (731 ± 100 pA | |
| ChR2 | 450 nm | ~10 mW/mm² | | 0.96 ± 0.12 | ms | No | 4.47 nA | HEK 293T |
| H134R ³ | | (470 nm) | | | | | | |
| ChETA ⁴ | 490 nm | ~10 | mW/mm² | 0.9 ± 0.1 | ms | No | steady state:peak | Hippocampal |
| | | | | | | | current ratio:0.6 ± | cell culture |
| | | | | | | | 0.04; (645 ± 47 pA | |
| ChrimsonR ⁵ | 590 nm | 4.6 | mW/mm² | 0.9 ± 0.1 | ms | No | ~300 pA | cultured neurons |
| mouse | 480 nm | 1015 photons | | >60 | s | 11-cis- | ~0.1 (ΔF/F) | HEK293-TRPC3 |
| melanopsin | | s⁻¹ cm⁻² | | | | retinaldehyde | Ca²⁺ response | cells |
| (Opn4) ⁶ | | (500 nm) | | | | | ampitude | |
| mouse | 488 nm | a white | | 60 | s | 11-cis retinal | ~0.25 (ΔF/F) | CHO cells |
| melanopsin | | fluorescent light | | | | | Ca²⁺ response | |
| (Opn4) and | | source (intensity | | | | | amplitude, the | |
| its mutants ⁷ | | undefined) | | | | | best mutant Opn4^{9A} | |
| hOpn4-human | 473 nm | 7 | mW/mm² | 20 | s | Unknown | ~2 (ΔF/F) | in vivo |
| melanopsin ⁸ | | | | | | | Ca²⁺ event | astrocytes |
| | | | | | | | frequence, but | |
| | | | | | | | no significant | |
| | | | | | | | change in Ca²⁺ | |
| | | | | | | | amplitude | |
| opto-α1AR ⁹ | 500 nm | 7 | mW/mm² | 60 | s | No | ~0.227 (ΔF/F) | HEK cells |
| | | | | | | | Ca²⁺ response | |
| | | | | | | | ampitude | |
| opto-α1AR ¹⁰ | 473 nm | 20 Hz, 45-ms light | | 5 | min | No | >20% increase in | in vitro |
| | | pulses, 5 mW | | | | | sIPSC frequency | astrocytes |
| human | 470 nm | 40 | mW/mm² | 25 | s | ATR | ~0.646 (ΔF/F) | HEK 293T |
| melanopsin | | | | | | | Ca²⁺ response | |
| | | | | | | | amplitude | |
| opto-α1AR | 510 nm | 7 | mW/mm² | 60 | s | No | ~0.5 (ΔF/F) | HEK 293T |
| | | | | | | | Ca²⁺ response | |
| | | | | | | | amplitude | |
| hM3Dq | | | | | | CNO | ~1.6 (ΔF/F) | HEK 293T |
| | | | | | | | Ca2+ event | |
| | | | | | | | frequence, but | |
| | | | | | | | no significant | |
| | | | | | | | change in Ca2+ | |
| | | | | | | | amplitude | |
| cOpn5 | 470 nm | 16 | μW/mm² | 10 | ms | No | ~3.0 (ΔF/F) | HEK 293T |
| | | | | | | | Ca²⁺ response | cells |
| | | | | | | | amplitude | |
| | 470 nm | 0.026 | μW/mm² | >2 | s | No | ~1 (ΔE/F) | HEK 293T |

| | |
|---|---|
| Ca²⁺ response | cells |
| amplitude | |

The performance of cOpn5 to that of opto-a1AR, a chimera GPCR engineered by mixing rhodopsin with G_{q}-coupled adrenergic receptor is compared. Following the protocol in a previous report¹⁴, it is found that very long exposure of strong illumination (60 s; 7 mW/mm²) is required to trigger a slow and small (˜0.5 ΔF/F) Ca²⁺ signal increase in opto-a1AR-expressing HEK 2931 cells, and 15 s illumination is inefficient (FIG. 4c, d). The performance of cOpn5 to that of opn4, a natural opsin which was reported as a tool for G_{q} signaling activating is also compared. It is found that long exposure of strong illumination (25 s; 40 mW/mm²) and additional retinal are required to trigger a slow (˜1 ΔF/F) Ca²⁺ signal increase in opn4-expressing HEK 293T cells (FIG. 4e, f). Therefore, compared with existing opsin-based tools (opto-a1AR and opn4), cOpn5 is much more light-sensitive (˜3 orders more sensitive), requires much shorter time exposure (10 ms vs. 60 s), and produces stronger responses.

The performance of cOpn5 to that of the popular G_{q}-coupled chemogenetic tool hM3Dq, which is activated by adding the exogenous small molecule ligand clozapine-N-oxide (CNO)³⁷⁻³⁹ is compared. Light-induced activation of cOpn5-expressing HEK 293T cells has a similar peak response amplitude of the Ca²⁺ signal as CNO-induced activation of hM3Dq-expressing HEK 293T cells. Meanwhile, cOpn5-expressing HEK 293T cells have faster and temporally more precise response, as well as more rapid recovery time than hM3Dq-expressing HEK 293T cells (FIG. 4g-i). These results indicate that cOpn5-mediated optogenetics are more controllable in temporal accuracy than those of hM3Dq.

cOpn5 optogenetics allows spatially precise control of cellular activity. Restricting brief light stimulation (63 ms) into a subcellular region of individual cOpn5-expressing HEK 293T cell results in the immediate activation of single cell. Interestingly, in high cell confluence area, the Ca²⁺ signals propagated to surrounding cells, thus suggesting intercellular communication among HEK 293T cells through a yet-to-identified mechanism (FIG. 3d, e). The findings are extended into primary cell cultures. cOpn5 is expressed in primary astrocyte cultures prepared from the neonatal mouse brain with AAV vectors for bicistronic expression of cOpn5 and the EGFP marker protein (FIG. 5a). Using the Calbryte 630 AM dye to monitor Ca²⁺ levels, it is found that blue light illumination of cOpn5-expressing astrocytes produces strong Ca²⁺ transients (˜8 ΔF/F) (FIG. 5b, c). If the light stimulation (63 ms) is precisely restricted to only subcellular region of an individual cOpn5-expressing astrocyte, Ca²⁺ signal propagation within the individual cell is observed (FIG. 3f). Resembling the tests in HEK 293T cells, wave-like propagation of Ca²⁺ signals from the stimulated astrocyte that proceeded gradually to more distal, non-stimulated, astrocytes is observed (FIG. 3g, h). These experiments thus demonstrate that cOpn5 optogenetics allows precise spatial control, and suggest that it may be useful to study the dynamics of astrocytic networks, which is initially discovered using neurochemical and mechanical stimulation^{40,41}.

FIG. 3 Shows that cOpn5 Sensitively Mediates Optical Control of G_{q} Signaling with High Temporal and Spatial Resolution.

a, Schematic diagram of selected wavelengths (365, 395, 470, 515, 561, 590, and 630 nm; left panel) and the amplitudes of Ca²⁺ signal of cOpn5-expressing HEK 293T cells in response to light stimulation with different wavelengths (2 s; 100 μW/mm²; right panel). Error bars indicate S.E.M.

b, The response magnitude under different duration of light stimulation (1, 5, 10, 20, or 50 ms; 16 μW/mm²; 470 nm). Error bars indicate S.E.M.

c, Time course of cOpn5-mediated Ca²⁺ signals under different light intensity (0, 4.8, 8, 16, or 32 μW/mm²; 10 ms; 470 nm; for 10 ms 16 μW/mm² stimulation, 10% peak activation=1.36±0.55 s; 90% peak activation=2.37±0.87 s; decay time τ=18.66±4.98 s, mean±S.E.M.; n=10 cells).

d, Images of light-induced (63 ms; 17 μW; arrow points to the stimulation region) Ca²⁺ signal propagation in cOpn5-expressing HEK 293T cells. Scale bar, 10 μm.

e, Pseudocolor images showing the process of Ca²⁺ signal propagation across time of d (frame N/(N−1)>1). Frame interval was 500 ms and each frame is counted once.

f, Images of light-induced Ca²⁺ signal propagation in a single cOpn5-expressing primary astrocyte stimulated in a subcellular region (stimulation size 4×4 μm² and frame interval 300 ms). Scale bar, 10 μm.

g, Images of light-induced Ca²⁺ signal propagation in cOpn5-expressing primary astrocytes. Scale bar, 10 μm.

h, Pseudocolor images showing process of Ca²⁺ signal propagation across time of g (frame N/(N−1)>1). Frame interval was 500 ms and each frame is counted once.

FIG. 4 Shows that cOpn5 Mediates More Rapid and Sensitive Response to Light than Opto-a1AR, hM3Dq or Opn4.

a, Time course of Ca²⁺ signal with light pulses (16 μW/mm²; 470 nm; 1, 5, 10, 20, or 50 ms).

b, The response magnitude under different light intensities (0, 4.8, 8, 16, or 32 μW/mm²) at 10 ms, 470 nm.

c, Pseudocolor images of the baseline and peak Ca²⁺ signals (ΔF/F0) in opto-a1AR-expressing HEK 293T cells. The medium buffer contains 10 μM all-trans-retinal. Scale bar, 30 μm.

d, Effect of 60 s light stimulation on the Ca²⁺ in opto-a1AR-expressing HEK 293T cells (n=15 cells; upper panel) and the lack of effect by 15 s light stimulation on Ca²⁺ signals (lower panel).

e, Pseudocolor images of the baseline and peak Ca²⁺ signals (ΔF/F0) in human OPN4-expressing HEK 293T cells. The medium buffer contains 10 μM all-trans-retinal. Scale bar, 30 μm.

f, Effect of 25 s light stimulation on the Ca²⁺ in OPN4-expressing HEK 293T cells within 10 uM ATR (n=12 cells; red line) and the lack of effect by without ATR on Ca²⁺ signals (black panel).

g, Effects of light stimulation on the Ca²⁺ signals in cOpn5-expressing HEK 293T cells. Upper panels show pseudocolor images of baseline and peak response. Lower panel shows the heat map of Ca²⁺ signals evoked by cOpn5-mediated optogenetic stimulation in HEK 293T cells expressing cOpn5 across 5 consecutive trials. Scale bar, 20 μm.

h, Effect of chemogenetic stimulation on the Ca²⁺ signals in hM3Dq-expressing HEK 293T cells.

i, Time courses of Ca²⁺ signals evoked by cOpn5-mediated optogenetic stimulation (10 s) and hM3Dq-mediated chemogenetic stimulation using CNO puff (100 nM; 10 s), respectively.

FIG. 5 Shows that cOpn5 Effectively Mediates the Activation of Astrocytes.

a, cOpn5 was expressed in cultured primary astrocytes using AAV-cOpn5-T2A-EGFP (green). Astrocyte identity was confirmed by GFAP immunostaining (red). Scale bar, 20 μm.

b, Pseudocolor images of the baseline and peak Ca²⁺ signals following light stimulation of cOpn5-expressing astrocytes. Scale bar, 20 μm.

c, Plot of Ca²⁺ signals and heat map representation of Ca²⁺ signals across trials (n=25 cells).

### Example 3 cOpn5 Optogenetic Activation of Astrocytes Induces Massive ATP Release and Neuron Activation In Vivo

The performance of cOpn5-mediated optogenetics in vivo is tested. Astrocytes represent an important population of non-excitable cells in the central nervous system, over which optogenetic tools have achieved only limited success to date⁴². ATP is known as a messenger for inter-astrocyte communication; however, the real-time impacts of intracellular Ca²⁺ on ATP release have not been visualized. Ultrasensitive GPCR Activation-Based ATP sensor GRAB_{ATP} is used to monitor changes in extracellular ATP levels. Specifically, cOpn5 and the GRAB_{ATP} sensor are expressed in the mouse S1 sensory cortex following the infusion of AAV vectors containing the GfaABC1D promoter (FIG. 6a), which is commonly used to drive gene expression in astrocytes.

Two-photon imaging of GRAB_{ATP} signals from head-fixed awake, behaving mice is performed (FIG. 6a). It is initially expected that, in addition to the 920 nm light from pulsed laser for two-photon imaging, blue light pulses would be required to stimulate ATP signals. Strikingly, the 920 nm light itself, delivers for imaging, triggered massive ATP flashes in the cOpn5- and GRAB_{ATP}-expressing mice, but not in mice that expressed the ATP sensor but lacked cOpn5 expression. Individual ATP flashes typically range in diameters of 20-100 μm and lasted for ˜1 min. The flash frequency gradually increases following ˜1 min of initial quiescence and peaked at the level of ˜50 flashes per min within the imaging area (640×640 μm²) in ˜5 min (FIG. 6b, c and FIG. 7a). Moreover, high-frequency ATP flashes also occurs in the repeated trials (FIG. 7b). In mice expressing GRAB_{ATP} alone, sporadic ATP events are observed (˜0.3 flashes per min within the imaging area); eight hours following the proinflammatory treatment of intraperitoneal lipopolysaccharide (LPS) injections, the ATP flash events increase nearly 6 fold of the basal condition (˜2 flashes per min) but exhibits rather stable frequency, which confirms that inflammation induces ATP release in the brain. Given that the observed ATP flash frequency in cOpn5-expressing mice is ˜25 times more than that by the proinflammatory treatment of non-cOpn5-expressing mice (FIG. 6d, FIG. 7c), it is demonstrated that cOpn5-mediated light activation of astrocytes induces continuous massive ATP release in vivo.

Astrocytes release ATP and other gliotransmitters also act on neuronal receptors to modulate neuron activity. Two-photon imaging of neuronal Ca²⁺ signals with cOpn5-mediated astrocytes activation from head-fixed awake, behaving mice, is performed (FIG. 6e). cOpn5-expressing cells (n=406) colocalize with GFAP-staining (n=397), but not GCaMP7b-expressing neurons (FIG. 6f). Raw trace examples and group data show that, compared the 15-20 min to 0-5 min, cOpn5-mediated astrocytes activation significantly elevates neuron activity (FIG. 6g, h and FIG. 7d). cOpn5 strictly expressed in astrocytes that demonstrated by the colocalization of cOpn5-expressing cells and GFAP staining signals which differ from GCaMP7b signals of neurons. We demonstrate that cOpn5-mediated light activation of astrocytes elevates the activities of surrounding neurons in vivo. Moreover, our data suggest that the long-wavelength (920 nm) light from pulsed laser for two-photon imaging is able to activate cOpn5, indicating the possibility of two-photon optogenetics for cOpn5.

FIG. 6 Shows that cOpn5-Mediated Activation of Astrocytes Induces Massive ATP Flashes and Neuron Activation In Vivo.

a, Schematic diagram of the experimental setup for in vivo two-photon imaging (920 nm) of ATP release following cOpn5-mediated astrocyte activation. Images show the expression of cOpn5 (red) in astrocytes and the expression of a GRAB_{ATP} sensor (green) in astrocytes within the mouse S1 cortex. Scale bar, 100 μm.

b, Astrocytic ATP flash events number across time (0-10 min) in a control mouse (no cOpn5 expression), a LPS-treated mouse (no cOpn5 expression), and a cOpn5-expressing mouse (Right).

c, Overall ATP flash events in a control mouse (no cOpn5 expression), a LPS-treated mouse (no cOpn5 expression), and a cOpn5-expressing mouse. The left column shows raw GRAB_{ATP} images at the basal level (before light delivery), the middle column shows GRAB_{ATP} signals at 5 min, and the right column shows pseudo-color-coded ATP flash events accumulated during 0-20 min.

d, Raster plot of astrocytic ATP flash events across time for the data shown in c.

e, Schematic diagram of the experimental setup for in vivo two-photon imaging (920 nm) of neuron calcium imaging following cOpn5-mediated astrocyte activation. Images show the expression of cOpn5 (red) in astrocytes and the expression of a GCaMP7b (green) in astrocytes within the mouse S1 cortex. Scale bar, 100 μm.

f, The cOpn5-expressing cells (red) were co-localized with 647 nm dye-counterstained GFAP cells (purple), GCaMP7b-expressing cells (green) are neurons. 406 red cells with 397 purple cells, Scale bar, 100 μm.

g, Temporal traces of ten individual GCaMP7b-expressing neurons in 0-5 min and 15-20 min, coupled with cOpn5-mediated astrocytes activation.

h, Calcium events analysis of GCaMP7b-expressing neurons in 0-5 min and 15-20 min, coupled with cOpn5-mediated astrocytes activation. N=193 neurons, ****P<0.0001, Unpaired t test.

FIG. 7 Shows that cOpn5 Mediates Persistent, Reliable ATP Release in Astrocytes and Activation of Surrounding Neurons.

a, Example of detected astrocytic ATP flash events in cOpn5-expressing mouse, with different colors indicating individual flashes.

b, ATP flash events (0-20 min) in cOpn5-expressing mouse in a repeat trial after 1 hour.

c, The quantification of ATP flash events number of the control, LPS, and cOpn5 groups. For the control-LPS comparison, **P=0.0066; LPS-cOpn5 comparison, **P=0.0031; control-cOpn5 comparison, ***P=0.0002, unpaired t tests.

d, Decoded spike rate analysis of GCaMP7b-expressing neurons in 0-5 min and 15-20 min, coupled with cOpn5-mediated astrocytes activation. N=193 neurons, ***P<0.0001, unpaired t test.

### Example 4 cOpn5 Optogenetics Activates Neurons and Modulates Animal Behaviors

The application of cOpn5-mediated optogenetics in neurons is explored. Whether cOpn5 could mediate light-induced Ca²⁺ signals is firstly examined. Using AAV and the pan-neuronal SYN promoter, cOpn5 and the genetically-encoded Ca²⁺ sensor jRGECOla are expressed in mouse cortical neurons (FIG. 8a). In brain slice preparations, application of blue light pulses (10 s; 100 μW/mm²; 473 nm) reliably evokes Ca²⁺ transients in neurons (FIG. 8b, c). Thus, cOpn5 also enables light-induced activation in neurons.

The effect of light-induced cOpn5 activation on the electrophysiological properties of neurons in slice preparations of the motor cortex, hippocampus, and dorsal striatum is next investigated (FIG. 8d). Two types of activation patterns are observed. In a majority of recorded neurons, blue light pulses induce a small depolarizing current (˜20 pA) in the voltage-clamp mode, and induce delayed-yet-vigorous firing of action potentials in the current-clamp mode (FIG. 8e, left, n=12 neurons). At higher frequencies of light pulses, cOpn5 drove more spikes with shorter latency (from ˜5 s to ˜3 s) after the initial light pulse, while the inward current is not significantly affected (FIG. 9a). In the other subset of neurons, brief light pulses rapidly evoked strong inward currents (100-1000 pA) and drove bursting firing of action potentials (FIG. 8e, right, n=6 neurons). Neurons are repeatedly stimulated with 10 ms/pulse at 10 Hz, and exhibit a non-attenuated mode in firing rate across repetitive trials of light stimulation (FIG. 9b). Of note, unlike generated by ChR2 optogenetics⁵³, the action potential evoked by cOpn5 photostimulation is not time-locked to light pulses.

Finally, the utility of cOpn5-mediated optogenetics for modulating animal behavior is assessed. The lateral hypothalamus (LH) is a brain center with known functions in reward processing and feeding behaviors^{54,55}. cOpn5 is expressed in the LH GABAergic neurons of VGAT-Cre mice and implanted optical fibers to deliver light pulses into the LH of freely behaving mice (FIG. 8g). Consistent with previous findings that activating LH GABA neurons drives feeding behavior⁵⁶, light stimulation (20 Hz; 5 ms/pulse; 473 nm; 0.75 mW output from the fiber tip) elicits a significant increase in food intake in cOpn5-expressing mice but not the EGFP-expressing control mice (FIG. 8h). A food-foraging behavior task is used to test the effect of cOpn5-mediated optogenetic activation of GABA neurons in the zona incerta (ZI) (FIG. 8i), a region known to drive compulsive eating⁵⁷. cOpn5-expressing mice, comparing with the EGFP-expressing mice, show a significantly increase in the time of foraging high fat food pellets upon repeated light stimulation (FIG. 8j). Electrophysiological recordings on LH and ZI cOpn5-expressing neurons are performed to characterize the response profiles. The injection sites and placement of optical fibers are confirmed by whole brain slices (FIG. 10a-c). Notably, mice maintained the behavior (feeding behavior or high-fat food foraging behavior) while the light is on, and immediately stopped the behavior when the light is off. Thus, cOpn5 is effective for rapidly, accurately, and reversibly modulating animal behavioral states.

FIG. 8 Shows that cOpn5-Mediated Optogenetics Changes Mouse Behaviors in a Neural Circuit-Dependent Manner.

a, Schematic diagram shows the experimental setup for optogenetic stimulation and Ca2+ imaging.

b, Pseudocolor images show Ca²⁺ signals before and after light stimulation (10 s; 100 μW/mm2; 473 nm). Scale bar, 10 μm.

c, Plots show Ca²⁺ signal traces of 6 individual neurons of b.

d, Schematic diagram depicts optogenetic stimulation and whole-cell patch-clamp recording of cOpn5-expressing neurons in the cortex, striatum and hippocampus.

e, Representative of two neurons that one exhibited strong, delayed firing of action potentials yet small sustained inward currents in response to light pulses (1 Hz, 5 s, 10 ms/pulse); the other one exhibited rapid membrane potential depolarization and large inward currents.

f, Firing rate of neurons increased after pulsed 473 nm light stimulation (1 Hz, 5 s) (***P=0.0005, n=18, unpaired t test).

g, Schematic diagram of the experimental setup for optogenetics and food intake assay. cOpn5-EGFP was expressed in GABAergic neurons within the lateral hypothalamus (LH) of VGAT-Cre mice. EGFP was expressed as a control.

h, Summary of light-induced (20 Hz; 5 ms/pulse; 473 nm; 0.75 mW output from the fiber tip), cOpn5-mediated activation of eating behaviors. ***P=0.0003; n.s., non-significant; n=6 mice; unpaired t test. Error bars indicate S.E.M.

i, Schematic diagram of the experimental setup for food foraging behavior. High-fat food pellets were used. cOpn5-EGFP was expressed in GABAergic neurons within the zonal incerta (ZI) of VGAT-Cre mice. EGFP was expressed as a control.

j, Summary of cOpn5-mediated food foraging behaviors. Foraging time percentage was calculated upon receiving light stimulation until the mouse found the hidden food. ****P<0.0001; n=6 mice; unpaired t test. Error bars indicate S.E.M.

FIG. 9 Shows that cOpn5-Mediated Optogenetics Reliably Activates Neurons.

a, One representative neuron in response to light pulses (5 s; 10 ms/pulse) at 1, 10, and 20 Hz.

b, Raw trace shows that cOpn5 mediated reliable and reproducible photoactivation of a neuron.

c, Summary of firing rates across repetitive trials of light stimulation.

FIG. 10 shows injection sites and the placement of optical fibers.

a, Images show the expression of EGFP control and bicistronic expression of cOpn5 in the LH (white dashed lines). Lesion sites and blue dashed lines indicate the placement of optical fibers. Scale bars, 500 μm.

b, The injection sites and optical fiber placement in the ZI. Scale bars, 500 μm.

c, Electrophysiological recordings on LH and ZI cOpn5-expressing neurons characterized cOpn5-mediated light activation.

### Example 5

Experiments description: the following table 9 is a partial list of cOpn5 orthologs from vertebrata tested in the present invention. Whole genes of all reported opsin5 orthologs from vertebrata (the vertebrates subphylum, including rotundia, cartilaginous fishes, bony fishes, Amphibia, reptila, ornitha and mammals) are synthetized, and expressed in HEK 293T cells. Calcium imaging with or without 470 nm blue light stimulation is performed to test the sensitivity of the opsin 5 orthologs in response to light. The time course of light-induced calcium signal reveal the activated degree of G_{q} signaling pathway and the sensitivity of these orthologs.

TABLE 9

| Entry | Entry name | Activity | Protein names | Gene names |
|---|---|---|---|---|
| E0R7P4 | E0R7P4_XENLA | | Opn5 (Opsin) | opn5.L opn5 |
| | | | | XELAEV_18028134mg |
| A0A455SGG5 | A0A455SGG5_9EUPU | | Opsin-5A | opn5a |
| A0A4Z2FX25 | A0A4Z2FX25_9TELE | | Opsin-5 | OPN5_5 EYF80_044932 |
| A0A4Z2FH27 | A0A4Z2FH27_9TELE | | Opsin-5 | OPN5_4 EYF80_049299 |
| A0A4Z2IDU8 | A0A4Z2IDU8_9TELE | | Opsin-5 | OPN5_3 EYF80_013671 |
| A0A4Z2H0H0 | A0A4Z2H0H0_9TELE | | Opsin-5 | OPN5_2 EYF80_030918 |
| A0A218USZ0 | A0A218USZ0_9PASE | | Opsin-5 | OPN5_1 |
| | | | | RLOC_00008660 |
| A0A4Z2FVH4 | A0A4Z2FVH4_9TELE | | Opsin-5 | Opn5_0 |
| | | | | EYF80_044930 |
| A0A4Z2HA58 | A0A4Z2HA58_9TELE | | Opsin-5 | OPN5_0 |
| | | | | EYF80_027087 |
| A0A218UGP1 | A0A218UGP1_9PASE | | Opsin-5 | OPN5_0 |
| | | | | RLOC_00005796 |
| G1L3V2 | G1L3V2_AILME | | Opsin 5 | OPN5 |
| A0A6P4X913 | A0A6P4X913_PANPR | | opsin-5 | OPN5 |
| A0A1S2ZDX4 | A0A1S2ZDX4_ERIEU | | opsin-5 | OPN5 |
| A0A2I4C032 | A0A2I4C032_9TELE | | opsin-5 | opn5 |
| U3JFW4 | U3JFW4_FICAL | | Opsin 5 | OPN5 |
| A0A2Y9NPU7 | A0A2Y9NPU7_DELLE | | opsin-5 | OPN5 |
| A0A1U7U6G6 | A0A1U7U6G6_CARSF | | opsin-5 | OPN5 |
| A0A6I9I544 | A0A6I9I544_VICPA | | opsin-5 | OPN5 |
| M3YLS7 | M3YLS7_MUSPF | | G_PROTEIN_RECEP_F1_2 | OPN5 |
| | | | domain-containing protein | |
| A0A5F9CCV1 | A0A5F9CCV1_RABIT | | Opsin 5 | OPN5 |
| A0A671EF51 | A0A671EF51_RHIFE | | Opsin 5 | OPN5 |
| A0A6P6I1D4 | A0A6P6I1D4_PUMCO | | opsin-5 | OPN5 |
| G3RKG7 | G3RKG7_GORGO | | Opsin 5 | OPN5 |
| G1NYV5 | G1NYV5_MYOLU | | Opsin 5 | OPN5 |
| A0A6J2L9P4 | A0A6J2L9P4_9CHIR | | opsin-5 | OPN5 |
| A0A2K6AXI4 | A0A2K6AXI4_MACNE | | Opsin 5 | OPN5 |
| A0A6J3JM90 | A0A6J3JM90_SAPAP | | opsin-5 | OPN5 |
| A0A452TE17 | A0A452TE17_URSMA | | Opsin 5 | OPN5 |
| A0A384C5D1 | A0A384C5D1_URSMA | | opsin-5 | OPN5 |
| A0A2K5U4B7 | A0A2K5U4B7_MACFA | | Opsin 5 | OPN5 |
| A0A2I3MZV4 | A0A2I3MZV4_PAPAN | | Opsin-5 | OPN5 |
| A0A2K5U4B3 | A0A2K5U4B3_MACFA | | Opsin 5 | OPN5 |
| Q6U736 | OPN5_HUMAN | reviewed | Opsin-5 (G-protein | OPN5 GPR136 |
| | | | coupled receptor 136) | PGR12 TMEM13 |
| | | | (G-protein coupled | |
| | | | receptor PGR12) | |
| | | | (Neuropsin) | |
| | | | (Transmembrane protein 13) | |
| F6UZB2 | F6UZB2_XENTR | | Opsin 5 | opn5 |
| A0A4W3IAF8 | A0A4W3IAF8_CALMI | | G_PROTEIN_RECEP_F1_2 | opn5 |
| | | | domain-containing protein | |
| A0A6P7HHM6 | A0A6P7HHM6_9TELE | | opsin-5 | opn5 |
| H3B1A3 | H3B1A3_LATCH | | G_PROTEIN_RECEP_F1_2 | OPN5 |
| | | | domain-containing protein | |
| A0A4W313H5 | A0A4W3I3H5_CALMI | | G_PROTEIN_RECEP_F1_2 | opn5 |
| | | | domain-containing protein | |
| A0A1S3MCD5 | A0A1S3MCD5_SALSA | | opsin-5 | opn5 |
| A0A4W4FPG5 | A0A4W4FPG5_ELEEL | | G_PROTEIN_RECEP_F1_2 | opn5 |
| | | | domain-containing protein | |
| A0A6P7LVJ1 | A0A6P7LVJ1_BETSP | | opsin-5 isoform X2 | opn5 |
| A0A6P7LVE3 | A0A6P7LVE3_BETSP | | opsin-5 isoform X1 | opn5 |
| A0A674IKC9 | A0A674IKC9_TERCA | | Opsin 5 | OPN5 |
| A0A674IMF3 | A0A674IMF3_TERCA | | Opsin 5 | OPN5 |
| F1NEY2 | F1NEY2_CHICK | | G_PROTEIN_RECEP_F1_2 | OPN5 |
| | | | domain-containing protein | |
| E6P6L8 | E6P6L8_DANRE | | Opsin 5 | opn5 |
| A0A671TVX9 | A0A671TVX9_SPAAU | | Opsin 5 | opn5 |
| A0A7M4FP40 | A0A7M4FP40_CROPO | | Opsin 5 | OPN5 |
| A0A671TVX4 | A0A671TVX4_SPAAU | | Opsin 5 | opn5 |
| A0A6I9Y3G3 | A0A6I9Y3G3_9SAUR | | opsin-5 | OPN5 |
| G1KNV3 | G1KNV3_ANOCA | | G_PROTEIN_RECEP_F1_2 | OPN5 |
| | | | domain-containing protein | |
| A0A493T549 | A0A493T549_ANAPP | | Opsin 5 | OPN5 |
| A0A6I9HELA | A0A6I9HELA_GEOFO | | opsin-5 isoform X2 | OPN5 |
| A0A218UPZ6 | A0A218UPZ6_9PASE | | Opsin-5 | OPN5 RLOC_00008263 |
| D8KW68 | D8KW68_ZONAL | | Opsin 5 | OPN5 |
| A0A663EIX5 | A0A663EIX5_AQUCH | | Opsin 5 | OPN5 |
| GINNA7 | GINNA7_MELGA | | Opsin 5 | OPN5 |
| A0A663EK31 | A0A663EK31_AQUCH | | Opsin 5 | OPN5 |
| A0A6J0Z1K0 | A0A6J0Z1K0_ODOVR | | opsin-5 | OPN5 |
| A0A6P3J431 | A0A6P3J431_BISBI | | opsin-5 | OPN5 |
| A0A2K5R3Y3 | A0A2K5R3Y3_CEBIM | | Opsin 5 | OPN5 |
| A0A671EF86 | A0A671EF86_RHIFE | | Opsin 5 | OPN5 mRhiFer1_012304 |
| A0A6I9ZSG3 | A0A6I9ZSG3_ACIJB | | opsin-5 | OPN5 |
| A0A2K6R8Q2 | A0A2K6R8Q2_RHIRO | | G_PROTEIN_RECEP_F1_2 | OPN5 |
| | | | domain-containing protein | |
| A0A4W2GZA3 | A0A4W2GZA3_BOBOX | | Opsin 5 | OPN5 |
| U3J4Q3 | U3J4Q3_ANAPP | | Opsin 5 | OPN5 |
| A0A6J2V8J5 | A0A6J2V8J5_CHACN | | opsin-5 | opn5 |
| A0A493T6P1 | A0A493T6P1_ANAPP | | Opsin 5 | OPN5 |
| A0A6J2J0L1 | A0A6J2J0L1_9PASS | | opsin-5 | OPN5 |
| A0A6P9CE92 | A0A6P9CE92_PANGU | | opsin-5 | OPN5 |
| A0A6J1V4P8 | A0A6J1V4P8_9SAUR | | opsin-5 | OPN5 |
| A0A288HLV3 | A0A288HLV3_ANSCY | | Opsin-5 | OPN5 |
| A0A151PID4 | A0A151PID4_ALLMI | | Opsin-5 | OPN5 Y1Q_0020212 |
| Q5RIV6 | Q5RIV6_DANRE | | Opsin 5 (Teleost neuropsin) | opn5 |
| D6RDV4 | D6RDV4_HUMAN | | Opsin-5 | OPN5 |
| J3KPQ2 | J3KPQ2_HUMAN | | Opsin-5 | OPN5 hCG_1642475 |
| F6XNY7 | F6XNY7_ORNAN | | Opsin 5 | OPN5 |
| A0A2K6FXK2 | A0A2K6FXK2_PROCO | | Opsin 5 | OPN5 |
| E2RPZ0 | E2RPZ0_CANLF | | Opsin 5 | OPN5 |
| A0A2K6V732 | A0A2K6V732_SAIBB | | Opsin 5 | OPN5 |
| A0A4X2K722 | A0A4X2K722_VOMUR | | Opsin 5 | OPN5 |
| A0A6P5KYE6 | A0A6P5KYE6_PHACI | | opsin-5 | OPN5 |
| A0A2K6FXJ4 | A0A2K6FXJ4_PROCO | | Opsin 5 | OPN5 |
| A0A4X2JZA4 | A0A4X2JZA4_VOMUR | | Opsin 5 | OPN5 |
| G1SX53 | G1SX53_RABIT | | Opsin 5 | OPN5 |
| A0A2U3WI94 | A0A2U3WI94_ODORO | | opsin-5 | OPN5 |
| A0A2K6V724 | A0A2K6V724_SAIBB | | Opsin 5 | OPN5 |
| A0A3Q7XKC9 | A0A3Q7XKC9_URSAR | | opsin-5 | OPN5 |
| A0A452RBH1 | A0A452RBH1_URSAM | | Opsin 5 | OPN5 |
| G1QVY1 | G1QVY1_NOMLE | | G_PROTEIN_RECEP_F1_2 | OPN5 |
| | | | domain-containing protein | |
| G1QVX6 | G1QVX6_NOMLE | | G_PROTEIN_RECEP_F1_2 | OPN5 |
| | | | domain-containing protein | |
| G3SJY5 | G3SJY5_GORGO | | Opsin 5 | OPN5 |
| A0A7N9CSX2 | A0A7N9CSX2_MACFA | | Opsin 5 | OPN5 |
| A0A384B2Q9 | A0A384B2Q9_BALAS | | opsin-5 | OPN5 |
| A0A2K6L978 | A0A2K6L978_RHIBE | | G_PROTEIN_RECEP_F1_2 | OPN5 |
| | | | domain-containing protein | |
| A0A2K6AXE7 | A0A2K6AXE7_MACNE | | Opsin 5 | OPN5 |
| A0A2J8P0S9 | A0A2J8P0S9_PANTR | | Opsin 5 | OPN5 |
| W5PR22 | W5PR22_SHEEP | | G_PROTEIN_RECEP_F1_2 | OPN5 |
| | | | domain-containing protein | |
| F7DJ88 | F7DJ88_CALJA | | G_PROTEIN_RECEP_F1_2 | OPN5 |
| | | | domain-containing protein | |
| A0A2K5R3Z8 | A0A2K5R3Z8_CEBIM | | Opsin 5 | OPN5 |
| F6PHB6 | F6PHB6_CALJA | | G_PROTEIN_RECEP_F1_2 | OPN5 |
| | | | domain-containing protein | |
| M3WMC9 | M3WMC9_FELCA | | Opsin 5 | OPN5 |
| A0A2K5L5D5 | A0A2K5L5D5_CERAT | | Opsin 5 | OPN5 |
| E1BNN4 | E1BNN4_BOVIN | | Opsin 5 | OPN5 |
| F6RFW7 | F6RFW7_MACMU | | Opsin 5 | OPN5 |
| A0A2J8RKP9 | A0A2J8RKP9_PONAB | | Uncharacterized protein | OPN5 |
| A0A3Q7RXX8 | A0A3Q7RXX8_VULVU | | opsin-5 | OPN5 |
| A0A2K5L5D9 | A0A2K5L5D9_CERAT | | Opsin 5 | OPN5 |
| H0WJY2 | H0WJY2_OTOGA | | Opsin 5 | OPN5 |
| A0A6P3ENQ6 | A0A6P3ENQ6_SHEEP | | opsin-5 | OPN5 |
| G3UA68 | G3UA68_LOXAF | | G_PROTEIN_RECEP_F1_2 | OPN5 |
| | | | domain-containing protein | |
| A0A6P5DVT1 | A0A6P5DVT1_BOSIN | | opsin-5 | OPN5 |
| A0A0D9RJS4 | A0A0D9RJS4_CHLSB | | Opsin 5 | OPN5 |
| I3LTK7 | I3LTK7_PIG | | Opsin 5 | OPN5 |
| A0A2K5Z564 | A0A2K5Z564_MANLE | | Opsin 5 | OPN5 |
| A0A5G2R7I1 | A0A5G2R7I1_PIG | | Opsin 5 | OPN5 |
| A0A6I9JGH7 | A0A6I9JGH7_CHRAS | | opsin-5 | OPN5 |
| A0A2K5Z517 | A0A2K5Z517_MANLE | | Opsin 5 | OPN5 |
| A0A452FM79 | A0A452FM79_CAPHI | | Opsin 5 | OPN5 |
| F6SJH5 | F6SJH5_HORSE | | Opsin 5 | OPN5 |
| A0A2R9BTW5 | A0A2R9BTW5_PANPA | | Opsin 5 | OPN5 |
| A0A2Y9FNI2 | A0A2Y9FNI2_PHYMC | | opsin-5 | OPN5 |
| A0A340WR35 | A0A340WR35_LIPVE | | opsin-5 | OPN5 |
| A0A6J2DJL3 | A0A6J2DJL3_ZALCA | | opsin-5 | OPN5 |
| A0A4X1UZM3 | A0A4X1UZM3_PIG | | G_PROTEIN_RECEP_F1_2 | OPN5 |
| | | | domain-containing protein | |
| A0A673TX31 | A0A673TX31_SURSU | | Opsin 5 | OPN5 |
| A0A341D5X7 | A0A341D5X7_NEOAA | | opsin-5 | OPN5 |
| A0A667FWA1 | A0A667FWA1_LYNCA | | Opsin 5 | OPN5 |
| A0A5B7H9S7 | A0A5B7H9S7_PORTR | | Opsin-5 | Opn5 E2C01_063173 |
| A0A337SC50 | A0A337SC50_FELCA | | Opsin 5 | OPN5 |
| H2RD19 | H2RD19_PANTR | | Opsin 5 | OPN5 |
| A0A2U3X849 | A0A2U3X849_LEPWE | | opsin-5 | OPN5 |
| G3THK6 | G3THK6_LOXAF | | G_PROTEIN_RECEP_F1_2 | OPN5 |
| | | | domain-containing protein | |
| A0A2U3V1E1 | A0A2U3V1E1_TURTR | | opsin-5 | OPN5 |
| A0A096NIY4 | A0A096NIY4_PAPAN | | Opsin-5 | OPN5 |
| A0A6P3PSZ2 | A0A6P3PSZ2_PTEVA | | opsin-5 | OPN5 |
| A0A2K5EFR2 | A0A2K5EFR2_AOTNA | | Opsin 5 | OPN5 |
| A0A3Q7QKC2 | A0A3Q7QKC2_CALUR | | opsin-5 | OPN5 |
| F7DVJ0 | F7DVJ0_MONDO | | Opsin 5 | OPN5 |
| A0A2K5EFU2 | A0A2K5EFU2_AOTNA | | Opsin 5 | OPN5 |
| A0A5F8H1F1 | A0A5F8H1F1_MONDO | | Opsin 5 | OPN5 |
| A0A2Y9H826 | A0A2Y9H826_NEOSC | | opsin-5 | OPN5 |
| G3W284 | G3W284_SARHA | | Opsin 5 | OPN5 |
| A0A3Q0CTY5 | A0A3Q0CTY5_MESAU | | opsin-5 | Opn5 |
| A0A6P5NS60 | A0A6P5NS60_MUSCR | | opsin-5 | Opn5 |
| H0V671 | H0V671_CAVPO | | Opsin 5 | OPN5 |
| I3M1B1 | I3M1B1_ICTTR | | Opsin 5 | OPN5 |
| Q7TQN6 | Q7TQN6_RAT | | G protein-coupled | Opn5 Gpr136 |
| | | | receptor 136 (Opsin 5) | |
| A0A287CZD4 | A0A287CZD4_ICTTR | | Opsin 5 | OPN5 |
| A0A1W6KZ83 | A0A1W6KZ83_9RODE | | Neuropsin | OPN5 |
| A0A6I9MCW1 | A0A6I9MCW1_PERMB | | opsin-5 | Opn5 |
| A0A6P3EVC3 | A0A6P3EVC3_OCTDE | | opsin-5 | Opn5 |
| A0A1S3FD42 | A0A1S3FD42_DIPOR | | LOW QUALITY | Opn5 |
| | | | PROTEIN: opsin-5 | |
| A0A6A4VE33 | A0A6A4VE33_AMPAM | | Opsin-5 | OPN5 FJT64_010458 |
| A0A4P2TKU6 | A0A4P2TKU6_PAROL | | Neuropsin | OPN5 |
| A0A670IDE8 | A0A670IDE8_PODMU | | G_PROTEIN_RECEP_F1_2 | OPN5 |
| | | | domain-containing protein | |
| A0A1U7S163 | A0A1U7S163_ALLSI | | opsin-5 | OPN5 |
| A0A670Y2N7 | A0A670Y2N7_PSETE | | Opsin 5 | OPN5 |
| K7FFW2 | K7FFW2_PELSI | | G_PROTEIN_RECEP_F1_2 | OPN5 |
| | | | domain-containing protein | |
| D9N3D0 | D9N3D0_COTJA | | Opsin 5 | OPN5 |
| Q6VZZ7 | OPN5_MOUSE | reviewed | Opsin-5 (G-protein coupled | Opn5 Gpr136 Pgr12 |
| | | | receptor 136) (G-protein | |
| | | | coupled receptor PGR12) | |
| | | | (Neuropsin) | |
| D8KWH6 | D8KWH6_ZONAL | | Opsin 5 | OPN5 |
| A0A674PPK4 | A0A674PPK4_TAKRU | | G_PROTEIN_RECEP_F1_2 | pn5 |
| | | | domain-containing protein | |
| A0A674HDZ6 | A0A674HDZ6_TAEGU | | G_PROTEIN_RECEP_F1_2 | OPN5 |
| | | | domain-containing protein | |
| H2V568 | H2V568_TAKRU | | G_PROTEIN_RECEP_F1_2 | opn5 |
| | | | domain-containing protein | |
| A0A6J0H1N3 | A0A6J0H1N3_9PASS | | opsin-5 | OPN5 |
| A0A672UEH1 | A0A672UEH1_STRHB | | Opsin 5 | OPN5 |
| A0A672UBX7 | A0A672UBX7_STRHB | | Opsin 5 | OPN5 |
| A0A6J0U919 | A0A6J0U919_9SAUR | | opsin-5 | OPN5 |
| A0A6J8E395 | A0A6J8E395_MYTCO | | OPN5 | MCOR_46347 |
| A0A6J7ZZ06 | A0A6J7ZZ06_MYTCO | | OPN5 | MCOR_1439 |
| A0A2J8RKQ7 | A0A2J8RKQ7_PONAB | | OPN5 isoform 1 | CR201_G0050220 |
| A0A2J8P0V4 | A0A2J8P0V4_PANTR | | OPN5 isoform 4 | CK820_G0007353 |
| A0A212D584 | A0A212D584_CEREH | | OPN5 | Celaphus_00014381 |

| Entry | Organism | Length |
|---|---|---|
| E0R7P4 | Xenopus laevis (African | 341 |
| | clawed frog) | |
| A0A455SGG5 | Ambigolimax valentianus | 425 |
| A0A4Z2FX25 | Liparis tanakae | 178 |
| | (Tanaka's snailfish) | |
| A0A4Z2FH27 | Liparis tanakae | 399 |
| | (Tanaka's snailfish) | |
| A0A4Z2IDU8 | Liparis tanakae | 396 |
| | (Tanaka's snailfish) | |
| A0A4Z2H0H0 | Liparis tanakae | 153 |
| | (Tanaka's snailfish) | |
| A0A218USZ0 | Lonchura striata | 348 |
| | domestica (Bengalese finch) | |
| A0A4Z2FVH4 | Liparis tanakae | 338 |
| | (Tanaka's snailfish) | |
| A0A4Z2HA58 | Liparis tanakae | 311 |
| | (Tanaka's snailfish) | |
| A0A218UGP1 | Lonchura striata | 417 |
| | domestica (Bengalese finch) | |
| G1L3V2 | Ailuropoda melanoleuca | 381 |
| | (Giant panda) | |
| A0A6P4X913 | Panthera pardus | 353 |
| | (Leopard) (Felis pardus) | |
| A0A1S2ZDX4 | Erinaceus europaeus | 353 |
| | (Western European hedgehog) | |
| A0A2I4C032 | Austrofundulus limnaeus | 353 |
| U3JFW4 | Ficedula albicollis | 357 |
| | (Collared flycatcher) | |
| | (Muscicapa albicollis) | |
| A0A2Y9NPU7 | Delphinapterus leucas | 362 |
| | (Beluga whale) | |
| A0A1U7U6G6 | Carlito syrichta (Philippine | 354 |
| | tarsier) (Tarsius syrichta) | |
| A0A6I9I544 | Vicugna pacos (Alpaca) | 353 |
| | (Lama pacos) | |
| M3YLS7 | Mustela putorius furo | 377 |
| | (European domestic ferret) | |
| | (Mustela furo) | |
| A0A5F9CCV1 | Oryctolagus cuniculus(Rabbit) | 366 |
| A0A671EF51 | Rhinolophus ferrumequinum | 380 |
| | (Greater horseshoe bat) | |
| A0A6P6I1D4 | Puma concolor (Mountain lion) | 353 |
| G3RKG7 | Gorilla gorilla gorilla | 382 |
| | (Western lowland gorilla) | |
| G1NYV5 | Myotis lucifugus (Little | 353 |
| | brown bat) | |
| A0A6J2L9P4 | Phyllostomus discolor (pale | 353 |
| | spear-nosed bat) | |
| A0A2K6AXI4 | Macaca nemestrina (Pig- | 354 |
| | tailed macaque) | |
| A0A6J3JM90 | Sapajus apella (Brown-capped | 354 |
| | capuchin) (Cebus apella) | |
| A0A452TE17 | Ursus maritimus (Polar bear) | 361 |
| | (Thalarctos maritimus) | |
| A0A384C5D1 | Ursus maritimus (Polar bear) | 353 |
| | (Thalarctos maritimus) | |
| A0A2K5U4B7 | Macaca fascicularis (Crab- | 354 |
| | eating macaque) (Cynomolgus | |
| | monkey) | |
| A0A2I3MZV4 | Papio anubis (Olive baboon) | 354 |
| A0A2K5U4B3 | Macaca fascicularis (Crab- | 382 |
| | eating macaque) (Cynomolgus | |
| | monkey) | |
| Q6U736 | Homo sapiens (Human) | 354 |
| F6UZB2 | Xenopus tropicalis (Western | 345 |
| | clawed frog) (Silurana | |
| | tropicalis) | |
| A0A4W3IAF8 | Callorhinchus milii (Ghost shark) | 340 |
| A0A6P7HHM6 | Parambassis ranga (Indian | 355 |
| | glassy fish) | |
| H3B1A3 | Latimeria chalumnae(Coelacanth) | 290 |
| A0A4W313H5 | Callorhinchus milii (Ghost shark) | 333 |
| A0A1S3MCD5 | Salmo salar (Atlantic salmon) | 328 |
| A0A4W4FPG5 | Electrophorus electricus | 333 |
| | (Electric eel) (Gymnotus | |
| | electricus) | |
| A0A6P7LVJ1 | Betta splendens (Siamese | 308 |
| | fighting fish) | |
| A0A6P7LVE3 | Betta splendens (Siamese | 365 |
| | fighting fish) | |
| A0A674IKC9 | Terrapene carolina triunguis | 372 |
| | (Three-toed box turtle) | |
| A0A674IMF3 | Terrapene carolina triunguis | 347 |
| | (Three-toed box turtle) | |
| F1NEY2 | Gallus gallus (Chicken) | 357 |
| E6P6L8 | Danio rerio (Zebrafish) | 352 |
| | (Brachydanio rerio) | |
| A0A671TVX9 | Sparus aurata (Gilthead | 357 |
| | sea bream) | |
| A0A7M4FP40 | Crocodylus porosus | 357 |
| | (Saltwater crocodile) | |
| | (Estuarine crocodile) | |
| A0A671TVX4 | Sparus aurata (Gilthead sea | 353 |
| | bream) | |
| A0A6I9Y3G3 | Thamnophis sirtalis | 277 |
| G1KNV3 | Anolis carolinensis (Green | 347 |
| | anole) (American chameleon) | |
| A0A493T549 | Anas platyrhynchos | 337 |
| | platyrhynchos (Northern mallard) | |
| A0A6I9HELA | Geospiza fortis (Medium | 354 |
| | ground-finch) | |
| A0A218UPZ6 | Lonchura striata domestica | 304 |
| | (Bengalese finch) | |
| D8KW68 | Zonotrichia albicollis | 354 |
| | (White-throated sparrow) | |
| A0A663EIX5 | Aquila chrysaetos chrysaetos | 343 |
| GINNA7 | Meleagris gallopavo (Wild turkey) | 358 |
| A0A663EK31 | Aquila chrysaetos chrysaetos | 370 |
| A0A6J0Z1K0 | Odocoileus virginianus texanus | 353 |
| A0A6P3J431 | Bison bison bison | 353 |
| A0A2K5R3Y3 | Cebus imitator (Panamanian | 382 |
| | white-faced capuchin) (Cebus | |
| | capucinus imitator) | |
| A0A671EF86 | Rhinolophus ferrumequinum | 354 |
| | (Greater horseshoe bat) | |
| A0A6I9ZSG3 | Acinonyx jubatus (Cheetah) | 353 |
| A0A2K6R8Q2 | Rhinopithecus roxellana | 354 |
| | (Golden snub-nosed monkey) | |
| | (Pygathrix roxellana) | |
| A0A4W2GZA3 | Bos indicus × Bos taurus | 355 |
| | (Hybrid cattle) | |
| U3J4Q3 | Anas platyrhynchos | 380 |
| | platyrhynchos (Northern mallard) | |
| A0A6J2V8J5 | Chanos chanos (Milkfish) | 355 |
| | (Mugil chanos) | |
| A0A493T6P1 | Anas platyrhynchos | 400 |
| | platyrhynchos (Northern mallard) | |
| A0A6J2J0L1 | Pipra filicauda (Wire-tailed | 354 |
| | manakin) | |
| A0A6P9CE92 | Pantherophis guttatus (Corn | 357 |
| | snake) (Elaphe guttata) | |
| A0A6J1V4P8 | Notechis scutatus (mainland | 357 |
| | tiger snake) | |
| A0A288HLV3 | Anser cygnoid (Swan goose) | 355 |
| A0A151PID4 | Alligator mississippiensis | 369 |
| | (American alligator) | |
| Q5RIV6 | Danio rerio (Zebrafish) | 352 |
| | (Brachydanio rerio) | |
| D6RDV4 | Homo sapiens (Human) | 382 |
| J3KPQ2 | Homo sapiens (Human) | 353 |
| F6XNY7 | Ornithorhynchus anatinus | 327 |
| | (Duckbill platypus) | |
| A0A2K6FXK2 | Propithecus coquereli | 354 |
| | (Coquerel's sifaka) | |
| | (Propithecus verreauxi coquereli) | |
| E2RPZ0 | Canis lupus familiaris (Dog) | 380 |
| | (Canis familiaris) | |
| A0A2K6V732 | Saimiri boliviensis boliviensis | 381 |
| | (Bolivian squirrel monkey) | |
| A0A4X2K722 | Vombatus ursinus (Common wombat) | 353 |
| A0A6P5KYE6 | Phascolarctos cinereus (Koala) | 355 |
| A0A2K6FXJ4 | Propithecus coquereli | 380 |
| | (Coquerel's sifaka) | |
| | (Propithecus verreauxi | |
| | coquereli) | |
| A0A4X2JZA4 | Vombatus ursinus (Common wombat) | 353 |
| G1SX53 | Oryctolagus cuniculus (Rabbit) | 353 |
| A0A2U3WI94 | Odobenus rosmarus divergens | 353 |
| | (Pacific walrus) | |
| A0A2K6V724 | Saimiri boliviensis boliviensis | 354 |
| | (Bolivian squirrel monkey) | |
| A0A3Q7XKC9 | Ursus arctos horribilis | 353 |
| A0A452RBH1 | Ursus americanus (American | 353 |
| | black bear) (Euarctos americanus) | |
| G1QVY1 | Nomascus leucogenys (Northern | 382 |
| | white-cheeked gibbon) | |
| | (Hylobates leucogenys) | |
| G1QVX6 | Nomascus leucogenys (Northern | 354 |
| | white-cheeked gibbon) | |
| | (Hylobates leucogenys) | |
| G3SJY5 | Gorilla gorilla gorilla | 354 |
| | (Western lowland gorilla) | |
| A0A7N9CSX2 | Macaca fascicularis (Crab-eating | 353 |
| | macaque) (Cynomolgus monkey) | |
| A0A384B2Q9 | Balaenoptera acutorostrata | 353 |
| | scammoni (North Pacific minke | |
| | whale) (Balaenoptera davidsoni) | |
| A0A2K6L978 | Rhinopithecus bieti (Black snub- | 333 |
| | nosed monkey) (Pygathrix bieti) | |
| A0A2K6AXE7 | Macaca nemestrina (Pig-ailed | 382 |
| | macaque) | |
| A0A2J8P0S9 | Pan troglodytes (Chimpanzee) | 354 |
| W5PR22 | Ovis aries (Sheep) | 377 |
| F7DJ88 | Callithrix jacchus (White- | 382 |
| | tufted-ear marmoset) | |
| A0A2K5R3Z8 | Cebus imitator (Panamanian | 354 |
| | white-faced capuchin) (Cebus | |
| | capucinus imitator) | |
| F6PHB6 | Callithrix jacchus (White- | 354 |
| | tufted-ear marmoset) | |
| M3WMC9 | Felis catus (Cat) (Felis | 353 |
| | silvestris catus) | |
| A0A2K5L5D5 | Cercocebus atys (Sooty mangabey) | 382 |
| | (Cercocebus torquatus atys) | |
| E1BNN4 | Bos taurus (Bovine) | 353 |
| F6RFW7 | Macaca mulatta (Rhesus macaque) | 354 |
| A0A2J8RKP9 | Pongo abelii (Sumatran orangutan) | 354 |
| | (Pongo pygmaeus abelii) | |
| A0A3Q7RXX8 | Vulpes vulpes (Red fox) | 353 |
| A0A2K5L5D9 | Cercocebus atys (Sooty mangabey) | 354 |
| | (Cercocebus torquatus atys) | |
| H0WJY2 | Otolemur garnettii (Small-eared | 352 |
| | galago) (Garnett's greater | |
| | bushbaby) | |
| A0A6P3ENQ6 | Ovis aries (Sheep) | 353 |
| G3UA68 | Loxodonta africana (African | 377 |
| | elephant) | |
| A0A6P5DVT1 | Bos indicus (Zebu) | 360 |
| A0A0D9RJS4 | Chlorocebus sabaeus (Green | 352 |
| | monkey) (Cercopithecus sabaeus) | |
| I3LTK7 | Sus scrofa (Pig) | 378 |
| A0A2K5Z564 | Mandrillus leucophaeus (Drill) | 382 |
| | (Papio leucophaeus) | |
| A0A5G2R7I1 | Sus scrofa (Pig) | 353 |
| A0A6I9JGH7 | Chrysochloris asiatica (Cape | 353 |
| | golden mole) | |
| A0A2K5Z517 | Mandrillus leucophaeus (Drill) | 354 |
| | (Papio leucophaeus) | |
| A0A452FM79 | Capra hircus (Goat) | 353 |
| F6SJH5 | Equus caballus (Horse) | 382 |
| A0A2R9BTW5 | Pan paniscus (Pygmy chimpanzee) | 353 |
| | (Bonobo) | |
| A0A2Y9FNI2 | Physeter macrocephalus (Sperm | 353 |
| | whale) (Physeter catodon) | |
| A0A340WR35 | Lipotes vexillifer (Yangtze | 353 |
| | river dolphin) | |
| A0A6J2DJL3 | Zalophus californianus | 353 |
| | (California sealion) | |
| A0A4X1UZM3 | Sus scrofa (Pig) | 378 |
| A0A673TX31 | Suricata suricatta (Meerkat) | 381 |
| A0A341D5X7 | Neophocaena asiaeorientalis | 353 |
| | asiaeorientalis (Yangtze | |
| | finless porpoise) (Neophocaena | |
| | phocaenoides subsp. | |
| | asiaeorientalis) | |
| A0A667FWA1 | Lynx canadensis (Canada lynx) | 376 |
| A0A5B7H9S7 | Portunus trituberculatus | 74 |
| | (Swimming crab) (Neptunus | |
| | trituberculatus) | |
| A0A337SC50 | Felis catus (Cat) (Felis | 376 |
| | silvestris catus) | |
| H2RD19 | Pan troglodytes (Chimpanzee) | 382 |
| A0A2U3X849 | Leptonychotes weddellii (Weddell | 365 |
| | seal) (Otaria weddellii) | |
| G3THK6 | Loxodonta africana (African | 360 |
| | elephant) | |
| A0A2U3V1E1 | Tursiops truncatus (Atlantic | 353 |
| | bottle-nosed dolphin) (Delphinus | |
| | truncatus) | |
| A0A096NIY4 | Papio anubis (Olive baboon) | 382 |
| A0A6P3PSZ2 | Pteropus vampyrus (Large | 353 |
| | flying fox) | |
| A0A2K5EFR2 | Aotus nancymaae (Ma's night | 382 |
| | monkey) | |
| A0A3Q7QKC2 | Callorhinus ursinus (Northern | 353 |
| | fur seal) | |
| F7DVJ0 | Monodelphis domestica (Gray | 346 |
| | short-tailed opossum) | |
| A0A2K5EFU2 | Aotus nancymaae (Ma's night | 354 |
| | monkey) | |
| A0A5F8H1F1 | Monodelphis domestica (Gray | 347 |
| | short-tailed opossum) | |
| A0A2Y9H826 | Neomonachus schauinslandi | 353 |
| | (Hawaiian monk seal) (Monachus | |
| | schauinslandi) | |
| G3W284 | Sarcophilus harrisii (Tasmanian | 355 |
| | devil) (Sarcophilus laniarius) | |
| A0A3Q0CTY5 | Mesocricetus auratus (Golden | 254 |
| | hamster) | |
| A0A6P5NS60 | Mus caroli (Ryukyu mouse) | 377 |
| | (Ricefield mouse) | |
| H0V671 | Cavia porcellus (Guinea pig) | 333 |
| I3M1B1 | Ictidomys tridecemlineatus | 353 |
| | (Thirteen-lined ground squirrel) | |
| | (Spermophilus tridecemlineatus) | |
| Q7TQN6 | Rattus norvegicus (Rat) | 534 |
| A0A287CZD4 | Ictidomys tridecemlineatus | 378 |
| | (Thirteen-lined ground squirrel) | |
| | (Spermophilus tridecemlineatus) | |
| A0A1W6KZ83 | Cricetulus barabensis (striped | 377 |
| | dwarf hamster) | |
| A0A6I9MCW1 | Peromyscus maniculatus bairdii | 377 |
| | (Prairie deer mouse) | |
| A0A6P3EVC3 | Octodon degus (Degu) (Sciurus | 353 |
| | degus) | |
| A0A1S3FD42 | Dipodomys ordii (Ord's kangaroo | 603 |
| | rat) | |
| A0A6A4VE33 | Amphibalanus amphitrite (Striped | 358 |
| | barnacle) (Balanus amphitrite) | |
| A0A4P2TKU6 | Paralichthys olivaceus (Bastard | 354 |
| | halibut) (Hippoglossus olivaceus) | |
| A0A670IDE8 | Podarcis muralis (Wall lizard) | 358 |
| | (Lacerta muralis) | |
| A0A1U7S163 | Alligator sinensis (Chinese | 350 |
| | alligator) | |
| A0A670Y2N7 | Pseudonaja textilis (Eastern | 385 |
| | brown snake) | |
| K7FFW2 | Pelodiscus sinensis (Chinese | 369 |
| | softshell turtle) (Trionyx | |
| | sinensis) | |
| D9N3D0 | Coturnix japonica (Japanese | 378 |
| | quail) (Coturnix coturnix | |
| | japonica) | |
| Q6VZZ7 | Mus musculus (Mouse) | 377 |
| D8KWH6 | Zonotrichia albicollis (White- | 354 |
| | throated sparrow) | |
| A0A674PPK4 | Takifugu rubripes (Japanese | 363 |
| | pufferfish) (Fugu rubripes) | |
| A0A674HDZ6 | Taeniopygia guttata (Zebra | 354 |
| | finch) (Poephila guttata) | |
| H2V568 | Takifugu rubripes (Japanese | 394 |
| | pufferfish) (Fugu rubripes) | |
| A0A6J0H1N3 | Lepidothrix coronata (blue- | 351 |
| | crowned manakin) | |
| A0A672UEH1 | Strigops habroptila (Kakapo) | 377 |
| A0A672UBX7 | Strigops habroptila (Kakapo) | 349 |
| A0A6J0U919 | Pogona vitticeps (central | 348 |
| | bearded dragon) | |
| A0A6J8E395 | Mytilus coruscus (Sea mussel) | 317 |
| A0A6J7ZZ06 | Mytilus coruscus (Sea mussel) | 235 |
| A0A2J8RKQ7 | Pongo abelii (Sumatran orangutan) | 382 |
| | (Pongo pygmaeus abelii) | |
| A0A2J8P0V4 | Pan troglodytes (Chimpanzee) | 353 |
| A0A212D584 | Cervus elaphus hippelaphus | 263 |
| | (European red deer) | |

### REFERENCES

1 Hauser, A. S., Attwood, M. M., Rask-Andersen, M., Schioth, H. B. & Gloriam, D. E. Trends in GPCR drug discovery: new agents, targets and indications. Nat Rev Drug Discov 16, 829-842, doi:10.1038/nrd.2017.178 (2017).
2 Wettschureck, N. & Offermanns, S. Mammalian G proteins and their cell type specific functions. Physiol Rev 85, 1159-1204, doi:10.1152/physrev.00003.2005 (2005).
3 Exton, J. H. Regulation of phosphoinositide phospholipases by hormones, neurotransmitters, and other agonists linked to G proteins. Annu Rev Pharmacol Toxicol 36, 481-509, doi:10.1146/annurev.pa.36.040196.002405 (1996).
4 Ritter, S. L. & Hall, R. A. Fine-tuning of GPCR activity by receptor-interacting proteins. Nature reviews Molecular cell biology 10, 819-830 (2009).
5 Kadamur, G. & Ross, E. M. Mammalian phospholipase C. Annu Rev Physiol 75, 127-154, doi:10.1146/annurev-physiol-030212-183750 (2013).
6 Gomez, J. L. et al. Chemogenetics revealed: DREADD occupancy and activation via converted clozapine. Science 357, 503-507, doi:10.1126/science.aan2475 (2017).
7 Adams, S. R. & Tsien, R. Y. Controlling cell chemistry with caged compounds. Annual review of physiology 55, 755-784 (1993).
8 Boyden, E. S., Zhang, F., Bamberg, E., Nagel, G. & Deisseroth, K. Millisecond-timescale, genetically targeted optical control of neural activity. Nat Neurosci 8, 1263-1268, doi:10.1038/nn1525 (2005).
9 Fenno, L., Yizhar, O. & Deisseroth, K. The Development and Application of Optogenetics. Annual Review of Neuroscience 34, 389-412, doi:10.1146/annurev-neuro-061010-113817 (2011).
Quadrato, G. et al. Cell diversity and network dynamics in photosensitive human brain organoids. Nature 545, 48-53 (2017).
11 Rost, B. R., Schneider-Warme, F., Schmitz, D. & Hegemann, P. Optogenetic tools for subcellular applications in neuroscience. Neuron 96, 572-603 (2017).
12 Tye, K. M. & Deisseroth, K. Optogenetic investigation of neural circuits underlying brain disease in animal models. Nature Reviews Neuroscience 13, 251-266 (2012).
13 Zhang, F. et al. The microbial opsin family of optogenetic tools. Cell 147, 1446-1457 (2011).
14 Airan, R. D., Thompson, K. R., Fenno, L. E., Bernstein, H. & Deisseroth, K. Temporally precise in vivo control of intracellular signalling. Nature 458, 1025-1029, doi:10.1038/nature07926 (2009).
Tichy, A. M., Gerrard, E. J., Sexton, P. M. & Janovjak, H. Light-activated chimeric GPCRs: limitations and opportunities. Curr Opin Struct Biol 57, 196-203, doi:10.1016/j.sbi.2019.05.006 (2019).
16 Koyanagi, M. & Terakita, A. Diversity of animal opsin-based pigments and their optogenetic potential. Biochim Biophys Acta 1837, 710-716, doi:10.1016/j.bbabio.2013.09.003 (2014).
17 Yau, K.-W. & Hardie, R. C. Phototransduction motifs and variations. Cell 139, 246-264 (2009).
18 Copits, B. A. et al. A photoswitchable GPCR-based opsin for presynaptic inhibition. Neuron 109, 1791-1809 e1711, doi:10.1016/j.neuron.2021.04.026 (2021).
19 Mahn, M. et al. Efficient optogenetic silencing of neurotransmitter release with a mosquito rhodopsin. Neuron 109, 1621-1635 e1628, doi:10.1016/j.neuron.2021.03.013 (2021).
20 Giiler, A. D. et al. Melanopsin cells are the principal conduits for rod-cone input to non-image-forming vision. Nature 453, 102-105 (2008).
21 Hankins, M. W., Peirson, S. N. & Foster, R. G. Melanopsin: an exciting photopigment. Trends in neurosciences 31, 27-36 (2008).
22 Hattar, S., Liao, H.-W., Takao, M., Berson, D. M. & Yau, K.-W. Melanopsin-containing retinal ganglion cells: architecture, projections, and intrinsic photosensitivity. Science 295, 1065-1070 (2002).
23 Xue, T. et al. Melanopsin signalling in mammalian iris and retina. Nature 479, 67-73 (2011).
24 Qiu, X. et al. Induction of photosensitivity by heterologous expression of melanopsin. Nature 433, 745-749 (2005).
Melyan, Z., Tarttelin, E. E., Bellingham, J., Lucas, R. J. & Hankins, M. W. Addition of human melanopsin renders mammalian cells photoresponsive. Nature 433, 741-745, doi:10.1038/nature03344 (2005).
26 Kojima, D. et al. UV-sensitive photoreceptor protein OPN5 in humans and mice. PLoS One 6, e26388, doi:10.1371/journal.pone.0026388 (2011).
27 Yamashita, T. et al. Opn5 is a UV-sensitive bistable pigment that couples with Gᵢ subtype of G protein. Proc Natl Acad Sci USA 107, 22084-22089, doi:10.1073/pnas.1012498107 (2010).
28 Zhang, K. X. et al. Violet-light suppression of thermogenesis by opsin 5 hypothalamic neurons. Nature 585, 420-425, doi:10.1038/s41586-020-2683-0 (2020).
29 Nakane, Y. et al. A mammalian neural tissue opsin (Opsin 5) is a deep brain photoreceptor in birds. Proceedings of the National Academy of Sciences 107, 15264-15268 (2010).
30 Nakane, Y., Shimmura, T., Abe, H. & Yoshimura, T. Intrinsic photosensitivity of a deep brain photoreceptor. Current Biology 24, R596-R597 (2014).
31 Rios, M. N., Marchese, N. A. & Guido, M. E. Expression of Non-visual Opsins Opn3 and Opn5 in the Developing Inner Retinal Cells of Birds. Light-Responses in Muller Glial Cells. Front Cell Neurosci 13, 376, doi:10.3389/fncel.2019.00376 (2019).
32 Mederos, S. et al. Melanopsin for precise optogenetic activation of astrocyte-neuron networks. Glia 67, 915-934 (2019).
33 Taniguchi, M. et al. Structure of YM-254890, a Novel G_{q}/11 Inhibitor from Chromobacterium sp. QS3666. Tetrahedron 59, 4533-4538, doi:10.1016/s0040-4020(03)00680-x (2003).
34 Hartwig, J. et al. MARCKS is an actin filament crosslinking protein regulated by protein kinase C and calcium-calmodulin. Nature 356, 618-622 (1992).
35 Zhang, F., Wang, L.-P., Boyden, E. S. & Deisseroth, K. Channelrhodopsin-2 and optical control of excitable cells. Nature methods 3, 785-792 (2006).
36 Lin, J. Y. A user's guide to channelrhodopsin variants: features, limitations and future developments. Experimental physiology 96, 19-25 (2011).
37 Gomez, J. L. et al. Chemogenetics revealed: DREADD occupancy and activation via converted clozapine. Science 357, 503-507 (2017).
38 Krashes, M. J. et al. Rapid, reversible activation of AgRP neurons drives feeding behavior in mice. The Journal of clinical investigation 121, 1424-1428 (2011).
39 Rogan, S. C., Roth, B. L. & Morrow, A. L. Remote Control of Neuronal Signaling. Pharmacological Reviews 63, 291-315, doi:10.1124/pr.110.003020 (2011).
40 Charles, A. C., Merrill, J. E., Dirksen, E. R. & Sandersont, M. J. Intercellular signaling in glial cells: calcium waves and oscillations in response to mechanical stimulation and glutamate. Neuron 6, 983-992 (1991).
41 Cornell-Bell, A. H., Finkbeiner, S. M., Cooper, M. S. & Smith, S. J. Glutamate induces calcium waves in cultured astrocytes: long-range glial signaling. Science 247, 470-473 (1990).
42 Zhang, F., Wang, L. P., Boyden, E. S. & Deisseroth, K. Channelrhodopsin-2 and optical control of excitable cells. Nat Methods 3, 785-792, doi:10.1038/nmeth936 (2006).
43 Davalos, D. et al. ATP mediates rapid microglial response to local brain injury in vivo. Nature neuroscience 8, 752-758 (2005).
44 Zhang, J.-m. et al. ATP released by astrocytes mediates glutamatergic activity-dependent heterosynaptic suppression. Neuron 40, 971-982 (2003).
45 Zhang, Z. et al. Regulated ATP release from astrocytes through lysosome exocytosis. Nature cell biology 9, 945-953 (2007).
46 Wu, Z. & Li, Y. New frontiers in probing the dynamics of purinergic transmitters in vivo. Neuroscience research 152, 35-43 (2020).
47 Wu, Z. et al., doi:10.1101/2021.02.24.432680 (2021).
48 Lawlor, P. A., Bland, R. J., Mouravlev, A., Young, D. & During, M. J. Efficient gene delivery and selective transduction of glial cells in the mammalian brain by AAV serotypes isolated from nonhuman primates. Molecular therapy 17, 1692-1702 (2009).
49 Lee, Y., Messing, A., Su, M. & Brenner, M. GFAP promoter elements required for region-specific and astrocyte-specific expression. Glia 56, 481-493 (2008).
50 Bal-Price, A., Moneer, Z. & Brown, G. C. Nitric oxide induces rapid, calcium-dependent release of vesicular glutamate and ATP from cultured rat astrocytes. Glia 40, 312-323, doi:10.1002/glia.10124 (2002).
51 Murakami, K., Nakamura, Y. & Yoneda, Y. Potentiation by ATP of lipopolysaccharide-stimulated nitric oxide production in cultured astrocytes. Neuroscience 117, 37-42 (2003).
52 Lezmy, J. et al. Astrocyte Ca(2+)-evoked ATP release regulates myelinated axon excitability and conduction speed. Science 374, eabh2858, doi:10.1126/science.abh2858 (2021).
53 Tian, L. et al. Imaging neural activity in worms, flies and mice with improved GCaMP calcium indicators. Nature methods 6, 875-881 (2009).
54 Jennings, J. H., Rizzi, G., Stamatakis, A. M., Ung, R. L. & Stuber, G. D. The inhibitory circuit architecture of the lateral hypothalamus orchestrates feeding. Science 341, 1517-1521 (2013).
55 Stuber, G. D. & Wise, R. A. Lateral hypothalamic circuits for feeding and reward. Nat Neurosci 19, 198-205, doi:10.1038/nn.4220 (2016).
56 Li, Y. et al. Hypothalamic Circuits for Predation and Evasion. Neuron 97, 911-924 e915, doi:10.1016/j.neuron.2018.01.005 (2018).
57 Zhang, X. & van den Pol, A. N. Rapid binge-like eating and body weight gain driven by zona incerta GABA neuron activation. Science 356, 853-859, doi:10.1126/science.aam7100 (2017).
58 Tsukamoto, H. & Terakita, A. Diversity and functional properties of bistable pigments. Photochemical & Photobiological Sciences 9, 1435-1443 (2010).
59 Ellis-Davies, G. C. Caged compounds: photorelease technology for control of cellular chemistry and physiology. Nature methods 4, 619-628 (2007).
60 Adams, S. R. & Tsien, R. Y. Controlling cell chemistry with caged compounds. Annu Rev Physiol 55, 755-784, doi:10.1146/annurev.ph.55.030193.003543 (1993).
61 Chen, T.-W. et al. Ultrasensitive fluorescent proteins for imaging neuronal activity. Nature 499, 295-300 (2013).
62 Jing, M. et al. An optimized acetylcholine sensor for monitoring in vivo cholinergic activity. Nature methods 17, 1139-1146 (2020).
63 Sun, F. et al. A genetically encoded fluorescent sensor enables rapid and specific detection of dopamine in flies, fish, and mice. Cell 174, 481-496. e419 (2018).
64 Wan, J. et al. A genetically encoded sensor for measuring serotonin dynamics. Nature Neuroscience 24, 746-752 (2021).
65 Zhao, Y. et al. An expanded palette of genetically encoded Ca2+ indicators. Science 333, 1888-1891 (2011).
66 Hochbaum, D. R. et al. All-optical electrophysiology in mammalian neurons using engineered microbial rhodopsins. Nat Methods 11, 825-833, doi:10.1038/nmeth.3000 (2014).
67 Sahel, J.-A. et al. Partial recovery of visual function in a blind patient after optogenetic therapy. Nature Medicine, 1-7 (2021).

1. An isolated light-sensitive opsin for activating G_{q} signaling and/or activating cells.

2. The isolated opsin of claim 1, which is an isolated opsin from an organism, its homologs, its orthologs, its paralogs, fragments or variants thereof having the activity of activating G_{q} signaling and/or activating cells.

3. The isolated opsin of claim 1, which shares at least 70%, at least 80%, at least 90%, at least 95%, at least 96%, at least 97%, at least 98%, or at least 99% identity to the wild type opsin in the organism, its homologs, its orthologs, its paralogs, fragments or variants thereof, and has the activity of activating G_{q} signaling and/or activating cells.

4. The isolated opsin of claim 1, which is an isolated opsin 5(Opn5) from an animal, its homologs, its orthologs, its paralogs, fragments or variants thereof having the activity of activating G_{q} signaling and/or activating cells.

5. The isolated opsin of claim 4, which shares at least 70%, at least 80%, at least 90%, at least 95%, at least 96%, at least 97%, at least 98%, or at least 99% identity to the wild type opsin 5(Opn5) in the animal, its homologs, its orthologs, its paralogs, fragments or variants thereof, and has the activity of activating G_{q} signaling and/or activating cells.

6. The isolated opsin of claim 2, wherein the organism is a vertebrate animal.

7. The isolated opsin of claim 6, wherein the vertebrate animal is an avian, a reptile, or a fish, an amphibian, or a mammal,
preferably, the animal is an avian, including but not limited to chicken, duck, goose, ostrich, emu, rhea, kiwi, cassowary, turkey, quail, chicken, falcon, eagle, hawk, pigeon, parakeet, cockatoo, makaw, parrot, perching bird (such as, song bird), jay, blackbird, finch, warbler and sparrow; or
preferably, the animal is a reptile including but not limited to lizard, snake, alligator, turtle, crocodile, and tortoise; or
preferably, the animal is a fish including but not limited to catfish, eels, sharks, and swordfish; or
preferably, the animal is an amphibian including but not limited to a toad, frog, newt, and salamander.

8. The isolated opsin of claim 4, wherein the isolated opsin 5(Opn5) is an isolated wild type opsin 5(Opn5) from the chicken, or fragments or variants thereof having the activity of activating G_{q} signaling and/or activating cells; or
the isolated opsin 5(Opn5) shares at least 70%, at least 80%, at least 90%, at least 95%, at least 96%, at least 97%, at least 98%, or at least 99% identity to the wild type opsin 5(Opn5) from the chicken, and has the activity of activating G_{q} signaling and/or activating cells.

9. The isolated opsin of claim 4, wherein the isolated opsin 5(Opn5) is an isolated wild type opsin 5(Opn5) from the turtle, or fragments or variants thereof having the activity of activating G_{q} signaling and/or activating cells; or
the isolated opsin 5(Opn5) shares at least 70%, at least 80%, at least 90%, at least 95%, at least 96%, at least 97%, at least 98%, or at least 99% identity to the wild type opsin 5(Opn5) from the turtle, and has the activity of activating G_{q} signaling and/or activating cells.

10. The isolated opsin of claim 4, wherein the isolated opsin 5(Opn5) has the amino acid sequence shown by SEQ ID NO:1 (cOpn5), or fragments or variants thereof having the activity of activating G_{q} signaling and/or activating cells; or
the isolated opsin 5(Opn5) shares at least 70%, at least 80%, at least 90%, at least 95%, at least 96%, at least 97%, at least 98%, or at least 99% identity to the amino acid sequence shown by SEQ ID NO:1 (cOpn5), and has the activity of activating G_{q} signaling and/or activating cells.

11. The isolated opsin of claim 4, wherein the isolated opsin 5(Opn5) has the amino acid sequence shown by SEQ ID NO:2 (tOpn5), or fragments or variants thereof having the activity of activating G_{q} signaling and/or activating cells; or
the isolated opsin 5(Opn5) shares at least 70%, at least 80%, at least 90%, at least 95%, at least 96%, at least 97%, at least 98%, or at least 99% identity to the amino acid sequence shown by SEQ ID NO:2 (tOpn5), and has the activity of activating G_{q} signaling and/or activating cells.

12. The isolated opsin of claim 4, wherein the light has a wavelength ranging range of 360 nm-520 nm, preferably, 450-500, more preferably, 460-480 nm, in particular, 470 nm.

13. An isolated nucleic acid encoding the isolated opsin of claim 1.

14. A chimeric gene comprising the sequence of the isolated nucleic acid in claim 13, operably linked to suitable regulatory sequences.

15. A vector comprising the isolated nucleic acid in claim 13,
preferably the vector is selected from a group consisting of a eukaryotic vector, a prokaryotic expression vector, a viral vector, or a yeast vector.

16. (canceled)

17. The vector of claim 16, which is a herpes virus simplex vector, a vaccinia virus vector, or an adenoviral vector, an adeno-associated viral vector, a retroviral vector, or an insect vector, preferably, wherein the vector is a recombinant AAV1, AAV2, AAV3, AAV4, AAV5, AAV6, AAV7, AAV8, AAV9, AAVS, AAVO or AAV10.

18. (canceled)

19. An isolated cell or a cell culture, comprising the isolated nucleic acid of claim 13.

20. (canceled)

21. A method of treating a disease or condition mediated by or involving activating G_{q} signaling and/or activating cells in a subject, comprising administering the isolated opsin of claim 1 to a subject in need thereof.

22. The method of claim 21, wherein the disease or condition includes diseases or conditions benefiting from activating G_{q} signaling and/or activating cells, for example, benefiting from the activation of astrocytes, strong ATP release, or elevating neuron activity,
preferably, the disease or condition includes diseases or conditions benefiting from activating islet cells, immune cells or nerve cells, more preferably from central neurons, astrocytes, glial cells, muscle cells, skeletal cells, endothelial cells, epithelial cells, nervous system cells, skin cells, lung cells, kidney cells and liver cells, cardiac cells, or vascular endothelial cells,
more preferably, the disease or condition is autoimmune disease, developmental disease, metabolic disease, mental disease, disease of respiratory system or cardiovascular disease.

23. (canceled)

24. (canceled)

25. The method of claim 21, wherein the method further comprises applying blue light having a wavelength range of 360 nm-550 nm, preferably, 450-500, more preferably, 460-480 nm, in particular, 470 nm, and/or
the method further comprises applying two-photon activation using light having a wavelength ≥920 nm.

26. (canceled)


SEQUENCE LISTING (12 sequences)

SEQ ID NO: 1 - protein, 357 aa, Gallus lafayetii
MSGMASDCNSSSQEEYLPHYVQQEDPFASKLSREADIIAGFYLTVIGILSTLGNGYVIFMSSKRKKKLRPAEIMTVNLAVCDLGISVVGKPFSIISFFSHRWIFGWMGCRWYGWAGFFFGCGSLITMTAVSLDRYLKICHLAYGTWLKRHHAFICLALIWAYATFWATVPFAGVGSYAPEPFGTSCTLDWWLAQASVAGQAFVLSILFFCLLFPTAVIVFSYVKIILKVKSSTKEVAHYDTRIQNSHILEMKLTKVAMLICAGFLIAWIPYAVVSVWSAFGQPDSVPIQFSVVPTLLAKSAAMYNPIIYQVIDCKFACCRSGGPKTLQKKSSLKESRMYTLSSHRDSAALSGTQLEV

SEQ ID NO: 2 - protein, 349 aa, Chelonia mydas
MASNHSTSPQDDHLPHYLQDEDPFASKLSREADIVAGFYLTIIGVLSTFGNGYVLFMSLKRKKKLKPAEIMTVNLAMCDLGISVVGKPFSIISFFSHRWVFGWLGCRWYGWAGFFFGCGSLITLTAVSLDRYLKICHLTYGTWLKRHHAFICLAIIWAYATFWATVPFAGLGNYAPEPFGTSCTLDWWLAQGSVAGQAFILNILFFCLLLPTAVIVFSYVKIIAKVKSSAKEVAHYDTRIQNSHVLEIKLTKVAMLICAGFLIAWIPYAVVSVWSAFGQPDSVPIQVSVIPTLLAKSAAMYNPIIYQVIDCKFSCCRSLQKKSSLKKSRLYTISGNREPTEMNETHLEL

SEQ ID NO: 3 - DNA, 89 bp, synthetic construct
  source 1..89: mol_type = other DNA
cgtgaggtaccggatcctctagaatgggcaagcccatccccaaccccctgctgggcctggacagcaccatgagtgggatggcatcggac

SEQ ID NO: 4 - DNA, 47 bp, synthetic construct
  source 1..47: mol_type = other DNA
tcgataagcttgatatcgaattcttagacttccagttgggttccgct

SEQ ID NO: 5 - DNA, 50 bp, synthetic construct
  source 1..50: mol_type = other DNA
tagagtcgagctcaagcttgccaccatgagtgggatggcatcggactgca

SEQ ID NO: 6 - DNA, 50 bp, synthetic construct
  source 1..50: mol_type = other DNA
aaccgcgggccctctagagcatatgttacttgtacagctcgtccatgccg

SEQ ID NO: 7 - DNA, 50 bp, synthetic construct
  source 1..50: mol_type = other DNA
acctccgctgctcgcggggtctagaatgagtgggatggcatcggactgca

SEQ ID NO: 8 - DNA, 50 bp, synthetic construct
  source 1..50: mol_type = other DNA
tatcgataagcttgatatcgaattcttacttgtacagctcgtccatgccg

SEQ ID NO: 9 - DNA, 51 bp, synthetic construct
  source 1..51: mol_type = other DNA
tacattatacgaagttatggcgcgccttattacttgtacagctcgtccatg

SEQ ID NO: 10 - DNA, 52 bp, synthetic construct
  source 1..52: mol_type = other DNA
atactttatacgaagttatgctagccaccatgagtgggatggcatcggactg

SEQ ID NO: 11 - DNA, 50 bp, synthetic construct
  source 1..50: mol_type = other DNA
gcatcacctccgctgctcgcggggtatgagtgggatggcatcggactgca

SEQ ID NO: 12 - DNA, 50 bp, synthetic construct
  source 1..50: mol_type = other DNA
tcaccatggtggcgaccgggggatctgggccaggattctcctcgacgtca